(19) United States
(12) Patent Application Publication
(10) Pub. No.: US 2020/0168291 A1
(43) Pub. Date: May 28, 2020

(54) PRIORITIZATION OF GENETIC MODIFICATIONS TO INCREASE THROUGHPUT OF PHENOTYPIC OPTIMIZATION

(71) Applicant: Zymergen Inc., Emeryville, CA (US)

(72) Inventors: Anupam Chowdhury, Oakland, CA (US); Peter Enyeart, Emeryville, CA (US); Michael Flashman, Eureka, CA (US); Alexander Glennon Shearer, San Francisco, CA (US); Kurt Thorn, San Francisco, CA (US)

(73) Assignee: Zymergen Inc., Emeryville, CA (US)

(21) Appl. No.: 16/619,809

(22) PCT Filed: Jun. 5, 2018
(86) PCT No.: PCT/US2018/036096

§ 371 Date: Dec. 5, 2019

Related U.S. Application Data
(60) Provisional application No. 62/516,053, filed on Jun. 6, 2017.

(51) Int. Cl. G16B 20/00; G16B 40/20; G16B 40/30; G06N 20/00; G06N 5/04

(52) U.S. Cl. CPC G16B 20/00; G16B 40/20; G06N 5/04; G06N 20/00; G16B 40/30

(57) ABSTRACT

Systems, methods and computer-readable media are provided for determining modifications to apply to genes within at least one microbial strain to improve phenotypic performance. The disclosure teaches accessing first phenotypic performance data based at least in part upon first gene modifications made to a first set of genes in at least one microbial strain; predicting second phenotypic performance of second gene modifications, based at least in part upon the first phenotypic performance data and at least one modification feature that is common to the first gene modifications and the second gene modifications; and prioritizing the second gene modifications to be applied to a second set of genes based at least in part upon the second phenotypic performance.

### CROSS-REFERENCE TO RELATED APPLICATIONS

This application claims priority to U.S. Provisional Application No. 62/516,053, filed Jun. 6, 2017, which is incorporated by reference in its entirety herein.

### BACKGROUND

### Field of the Disclosure

The disclosure relates generally to the fields of metabolic and genomic engineering, and more particularly to the field of high throughput (“HTP”) genetic modification of microbial strains to produce products of interest.

### Description of Related Art

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Genetically optimizing an organism to exhibit a desired phenotype is a well-known problem. The two main sub-problems that confront the metabolic engineer are: (1) of all the possible modifications that might be made to the organism, which should be attempted to maximize output of the desired compound; and (2) once a set of modifications has been decided on, in which order should they be performed to maximize the rate of progress?

Conventionally, the genes targeted for modification are those genes that are judged to be “on-pathway,” i.e., the genes for the metabolic enzymes known to be part of, or branching into or off of, the biosynthetic pathway for the molecule of interest (Keasling, J D. “Manufacturing molecules through metabolic engineering.” Science, 2010). Methods such as flux balance analysis (“FBA”) (Segre et al, “Analysis of optimality in natural and perturbed metabolic networks.” PNAS, 2002) are known that can automate the discovery of such genes. While it is clear that modifications to the genes identified this way often result in improved strain performance, it is also true that even the simplest microbes remain poorly understood. Applicants have discovered that modification of other genes not directly involved in such pathways can produce significant improvements to strain performance, suggesting the need to investigate other genes in the genome. However, modifying every gene in a genome, even the relatively small genomes of bacteria, remains an expensive and time-consuming endeavor. It is desired to speed up the process of identifying target genes and the modifications to be made to those target genes that are useful for optimizing the production of a molecule of interest.

### SUMMARY OF THE DISCLOSURE

Embodiments of the present disclosure overcome the drawbacks of conventional techniques by prioritizing the genes to be modified and the modifications to be made to those genes.

The basic approach of some embodiments of the disclosure is to divide the genes of the genome into priority levels, called “shells,” and then implement planned modifications on those shells in order. In embodiments, shells can be designed by algorithms that leverage existing datasets relating to metabolic networks, gene ontology, or the performance of modifications made to corresponding genes in another organism or with another target product, or both, in mind. The exact nature of the modifications to be performed can also be prioritized; for example, changing to weaker promoters tends to provide fewer improvements than stronger promoters, which, according to experiments performed by the inventors, provide fewer improvements than medium-strength promoters. In some instances, swapping in weak promoters may down-regulate the production of compounds that interfere with production of the desired product of interest. As an optimization effort progresses, data can be collected about which classes of modifications provide the best performance improvements, which can then be fed back in an “online,” dynamic, iterative fashion for prioritizing the next round of modifications. Such datasets can also be applied toward prioritizing the types of gene modifications (e.g., promoter or SNP modifications) for optimizations of new phenotypes and/or organisms.

The shell metaphor for target prioritization of genes to be modified is based on the hypothesis that only a handful of primary genes are responsible for most of a particular aspect of a host cell's performance (e.g., production of a single biomolecule). These primary genes are located at the core of the shell, followed by secondary effect genes in the second layer, tertiary effects in the third shell, and so on. For example, in one embodiment the core of the shell may comprise genes encoding biosynthetic enzymes directly involved in a selected metabolic pathway (e.g., production of citric acid). Genes located on the second shell might comprise genes encoding for other enzymes within the biosynthetic pathway responsible for product diversion or feedback signaling. Third tier genes under this illustrative metaphor would likely comprise regulatory genes responsible for modulating expression of the biosynthetic pathway, or for regulating general carbon flux within the host cell.

Embodiments of the disclosure provide systems, methods, and computer-readable media for developing a prioritization for applying modifications to genes within at least one microbial strain to improve phenotypic performance. Embodiments of the disclosure provide a computer-implemented method, as well as systems and non-transitory computer-readable media for implementing the method. According to embodiments, the method comprises accessing first phenotypic performance data based at least in part upon first gene modifications made to a first set of genes in at least one microbial strain; predicting second, predicted phenotypic performance of second gene modifications based at least in part upon the first phenotypic performance data and at least one modification feature that is common to the first gene modifications and the second gene modifications; and prioritizing the second gene modifications to be applied to a second set of genes based at least in part upon the second phenotypic performance. Based at least in part upon the prioritizing, second gene modifications may be applied to genes within at least one microbial strain. A modification feature is a parameter considered to be of possible utility in predictive modeling, e.g., machine learning. Modification features may be expressed as categorical features (e.g., a type), continuous (e.g., a number), or ordinal features (e.g., discrete groups, such as better or worse).

According to embodiments of the disclosure, the gene modifications and the at least one modification feature may relate to the genes to be modified or to the types of modifications to be made to those genes. For example, the at least one modification feature may include class including ontological class, such as class related to GO classification, or to the type of modification, such as a promoter swap (e.g., a promoter modification, including insertion, deletion, or replacement of a promoter), or a SNP (single nucleotide polymorphism) swap (e.g., a single base pair modification, including insertion, deletion or replacement of a single base pair), as described in copending U.S. patent application Ser. No. 15/396,230, U.S. Publication No. US20170159045, filed Dec. 30, 2016, which is incorporated by reference herein in its entirety.

The modification feature may be related to the strength of the promoter, such as weak, strong, or medium strength. Experiments by the inventors have shown instances where medium strength promoters generated a greater likelihood of performance (e.g., yield, productivity) improvement by the microbial strain than did weak or strong promoters. Thus, embodiments of the disclosure may weight medium-strength promoters more heavily than strong or weak promoters into the predicted phenotypic performance. Embodiments of the disclosure may weight weak promoters less heavily than strong and medium-strength promoters.

In general, embodiments may weight known beneficial effects more heavily into the predicted phenotypic performance than lesser effects. Conversely, embodiments may assign low weighting to known negative or less beneficial effects in the predicted phenotypic performance than more beneficial effects. As another example, in embodiments predicting second phenotypic performance of second gene modifications is based at least in part upon at least one modification feature including modifications of one or more types (e.g., promoter swap, SNP swap) to at least two genes in a strain. In this manner, the method accounts for epistatic effects arising from the phenotypic effects of making two or more gene modifications to the same strain. In such embodiments, predicting may more heavily weight, into the predicted phenotypic performance, modifications of one or more types that yield positive epistatic effects.

In embodiments, the at least one modification feature includes different levels of abstraction within a gene ontology classification. In embodiments, the at least one modification feature includes classification based upon metabolic network. In embodiments, the second set of genes includes no genes within the first set of genes. In embodiments, genes within the second set of genes are each a member of multiple classes, and a composite performance prediction for a given gene can be generated from the combination of predictions applying to each class to which it belongs. In embodiments, genes within the second set of genes share membership in at least one common class, and such genes are all assigned the same predicted performance if the common class is the only class to which each gene belongs. In embodiments, genes within the second set of genes may each be a member of only a single class. In embodiments, genes in the first and second sets may share class membership with each other and such genes may each belong to multiple classes.

In embodiments, the at least one modification feature includes first ontological classes from a first classification system and second ontological classes from a second classification system. If, for example, a gene is a member of multiple classes from different classification systems (e.g., GO, KEGG, gene or gene-product sequence similarity, protein domain) and those classes have been observed or predicted to yield performance improvements, then the method may favorably weight the predicted phenotypic performance of that gene as a candidate for modification (thereby increasing its chance of being assigned a high priority), according to embodiments of the disclosure.

In embodiments, the at least one modification feature includes a characteristic of the product produced by at least one microbial strain. For example, the characteristic of the product may be related to the same metabolic pathway or ontological class. If the first set or a gene from the first set are associated with a performance improvement, then it is likely that a gene from the second set along the same metabolic pathway or within the same ontological class would also give rise to a performance improvement. Thus, the method may favorably weight the predicted phenotypic performance of that gene as a candidate for modification (thereby increasing its chance of being assigned a high priority), according to embodiments of the disclosure.

Alternatively, if multiple strain-product combinations are used as modification features of phenotypic performance data, characteristics of the product may be used to weight the relevance of data relating to an input strain-product combination to the target strain-product combination. Inputs that share more characteristics with the target product are more likely to yield useful predictions. In embodiments, those product characteristics may include number of constituent atoms, structure, atomic content, being produced from closely related (either by content or distance to nearest common precursor) metabolic pathways, or the like, with the first product.

In embodiments, predicting second phenotypic performance may employ genes from the first set of genes as a training set in a machine learning predictive model to predict the second phenotypic performance of the second gene modifications.

In embodiments, predicting second phenotypic performance comprises predicting per-class enrichment probabilities for the second gene modifications based at least in part upon the first, observed phenotypic performance data, and prioritizing the second, predicted gene modifications based at least in part upon a ranking of the predicted per-class enrichment probabilities. Embodiments of the disclosure may prioritize at least one candidate gene for testing within a class if the predicted enrichment for the class exceeds a threshold enrichment.

Applicants have further surprisingly discovered that individual gene performance can be context dependent, i.e., that the ability of a modification to a gene to improve strain performance can depend on the genetic makeup (including previously introduced modifications) of the strain. For example, whereas a particular gene modification may initially be predicted to have no, little, or even a negative effect on strain performance, the introduction of the same modification in a different genetic background can produce a different and even opposite effect. Thus, in embodiments of the disclosure, the method may comprise iteratively updating prioritization of subsets of the second gene modifications to be applied to subsets of genes within the second set of genes based upon phenotypic performance data observed from iterative application of one or more gene modifications of the second gene modifications to genes within the second set of genes. Such iterative updating may comprise obtaining updated phenotypic performance data based at least in part upon application of one or more gene modifications of the second gene modifications to genes within the second set of genes, predicting updated second phenotypic performance of a subset of the second gene modifications based at least in part upon the updated first phenotypic performance data and at least one modification feature, and prioritizing the subset of the second gene modifications to be applied to a subset of genes within the second set of genes based at least in part upon the updated second phenotypic performance. Note that the application of one or more gene modifications of the second gene modifications to genes within the second set of genes effectively moves those modified genes from within the second set of genes to the first set of genes, for which performance data may now be obtained, according to embodiments of the disclosure.

In embodiments, the at least one modification feature relates to a characteristic of microbial strain. Such features may include phylogenetic or taxonomic features, including genomic sequence similarity, domain (Archaea, Bacteria, or Eukarya), Gram positive or negative (for the bacteria), genus, species, and the like; ecological and physiological features, including features of the native environment (e.g., pH, temperature, salinity, pressure), metabolic features (e.g., preferred growth substrates, possible growth substrates, waste products), and the like; or other features. For example, if a modification to a set of genes in a first strain provides a performance improvement, then it is likely that a similar modification to a similar set of genes in a similar, second strain would also give rise to a performance improvement. “Similar set of genes” here may be defined as, e.g., genes belonging to the same gene ontology class, belonging to a metabolic pathway having the same product, sequence similarity, similarity in expression profile or regulation, or the like. “Similar” strains may be characterized by phylogentic similarity, similarlity in genetic lineage; whether the strains are prokaryotic or eukaryotic, consume similar feedstock, produce the similar metabolites, or are similar in other modification features. Thus, the method may favorably weight the predicted phenotypic performance of genes within that similar set in the second strain as candidates for modification by the same or a similar modification, according to embodiments of the disclosure.

In embodiments, the second set of genes resides within at least one microbial strain different from the at least one microbial strain in which the first set of genes resides. In those embodiments and others, the first phenotypic performance data may relate to at one or more characteristics of a first product produced by the at least one microbial strain, and the second, predicted phenotypic performance may relate to one or more characteristics of a second product that is different from the first product, and produced by the same strain or another strain sharing common features. In embodiments, the second product may share common features, such as number of constituent atoms, structure, atomic content, being produced from closely related (either by content or distance to nearest common precursor) metabolic pathways, or the like, with the first product.

### BRIEF DESCRIPTION OF THE DRAWINGS

FIG. 1 illustrates a client-server computer system for implementing embodiments of the disclosure.

FIG. 2 illustrates the fraction of modifications whose level of improvement exceeds a noise threshold for phenotypes representing productivity and yield of a target product across different promoter strengths, according to embodiments of the disclosure.

FIG. 3 illustrates a modification of FIG. 2, aggregated by library goal—diversification or consolidation.

FIG. 4 illustrates subsets of the data from FIG. 2 that are designed to even out the bias in frequency across the different promoter levels, according to embodiments of the disclosure.

FIG. 5 illustrates the fraction of modifications whose level of improvement is above a noise threshold for phenotypes of productivity and yield of a target product according to selection by a skilled human or an algorithm (FBA), aggregated by library goal, according to embodiments of the disclosure.

FIG. 6 illustrates an example of a subgraph from the Gene Ontology, showing gene classes enriched for improved yield.

FIG. 7 illustrates a breakdown of genes in the enriched GO Slims of Table 2.

FIG. 8 illustrates the breakdown of the subset of genes in enriched GO slims whose modification via promoter swap has been demonstrated to improve a desired phenotype, according to embodiments of the disclosure.

FIG. 9 is a flowchart illustrating a method for prioritizing modifications for application to genes within at least one microbial strain to improve phenotypic performance.

FIG. 10 illustrates a cloud computing environment according to embodiments of the disclosure.

FIG. 11 illustrates an example of a computer system that may be used to execute program code to implement embodiments of the disclosure

FIG. 12 is a diagram of the layout of the tables of FIGS. 12A-12L, which together form a table illustrating attributes involved in the production of particular amino acid in a particular microbial host organism.

### DETAILED DESCRIPTION

The present description is made with reference to the accompanying drawings, in which various example embodiments are shown. However, many different example embodiments may be used, and thus the description should not be construed as limited to the example embodiments set forth herein. Rather, these example embodiments are provided so that this disclosure will be thorough and complete. Various modifications to the exemplary embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

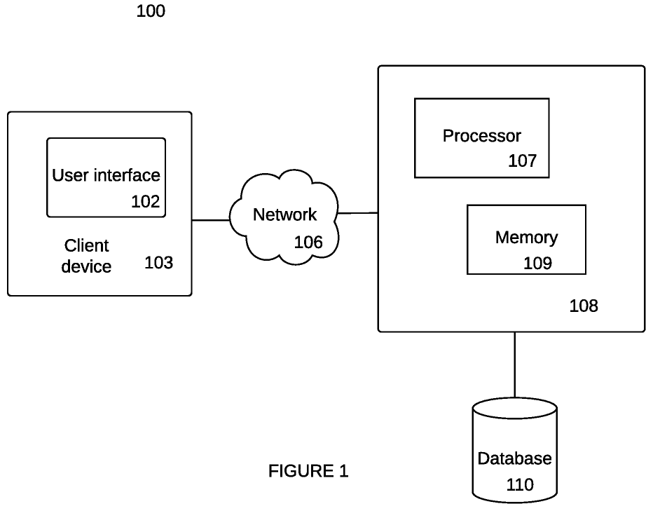

FIG. 1 illustrates a distributed system 100 of embodiments of the disclosure. A user interface 102 includes a client-side interface such as a text editor or a graphical user interface (GUI). The user interface 102 may reside at a client-side computing device 103, such as a laptop or desktop computer. The client-side computing device 103 is coupled to one or more servers 108 through a network 106, such as the Internet.

The server(s) 108 are coupled locally or remotely to one or more databases 110, which may include one or more corpora of libraries including data such as genome data, genetic modification data (e.g., promoter ladders), and phenotypic performance data that may represent microbial strain performance in response to genetic modifications.

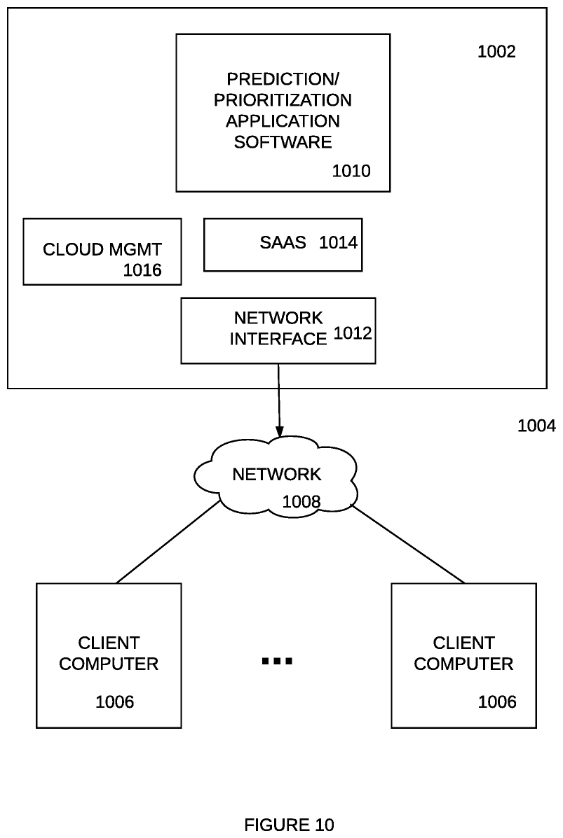

In embodiments, the server(s) 108 includes at least one processor 107 and at least one memory 109 storing instructions that, when executed by the processor(s) 107, predict phenotypic performance of gene modifications and prioritize their application to genes, thereby acting as a “prioritization engine” according to embodiments of the disclosure. Alternatively, the software and associated hardware for the prioritization engine may reside locally at the client 103 instead of at the server(s) 108, or be distributed between both client 103 and server(s) 108. In embodiments, all or parts of the prioritization engine may run as a cloud-based service, depicted further in FIG. 10.

The database(s) 110 may include public databases, as well as custom databases generated by the user or others, e.g., databases including molecules generated via synthetic biology experiments performed by the user or third-party contributors. The database(s) 110 may be local or remote with respect to the client 103 or distributed both locally and remotely.

The most conceptually simple way to modulate flux and yield to a desired molecule is by changing the amounts of gene products that affect that flux by changing the strength of the relevant gene promoters. This can be accomplished systematically by building a promoter ladder, a collection of promoters that can be applied to any gene and that have a range of strengths from weak to strong. Ideally, the promoters placed in the ladder have been shown to lead to highly variable expression across multiple genomic loci, but the only requirement is that they perturb gene expression in some way.

The promoter ladders are further described in International Application Serial No. PCT/US16/65464, WO2017/100376, filed on Dec. 7, 2016, which is incorporated by reference in its entirety. In embodiments, promoter ladders are created by: identifying natural, native, or wild-type promoters associated with the target gene of interest and then mutating at least one promoter to derive multiple mutated promoter sequences. Each of these mutated promoters is tested for effect on target gene expression. In some embodiments, the edited promoters are tested for expression activity across a variety of conditions, such that each promoter variant's activity is documented/characterized/annotated and stored in a database. The resulting edited promoter variants are subsequently organized into “ladders” arranged based on the strength of their expression (e.g., with highly expressing variants near the top, and attenuated expression near the bottom, therefore leading to the term “ladder”).

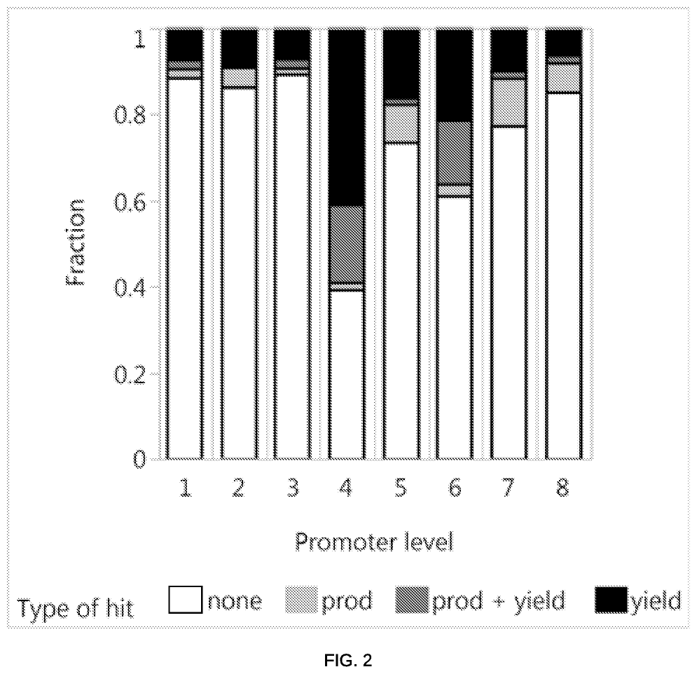

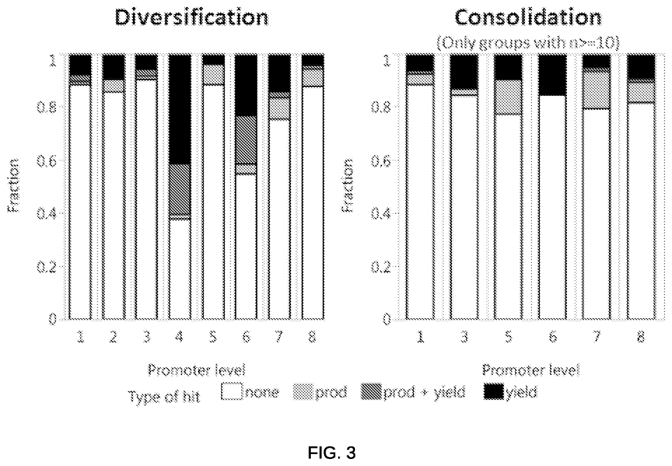

The process of changing the native promoter to one of the promoters from the ladder is called “promoter swapping.” Experimental data indicates that medium and strong promoter swaps are more likely to result in improvements in the desired phenotype than weak promoter swaps as shown in FIG. 2.

FIG. 2 illustrates the fraction of modifications (here, promoter swaps) whose level of improvement is above a noise threshold for phenotypes representing productivity and yield of a target product across different promoter strengths (1 being the weakest and 8 being the strongest). Note that the number of attempted modifications is not even across promoters; the total counts, in order from strength 1 to 8, are 532, 22, 422, 61, 68, 415, 108, and 3274.

There are several ways to define “weak,” “medium,” and “strong” in reference to promoters. In the embodiments here, these definitions are best understood within the context of an eight-promoter ladder designed to cover the majority of feasible expression levels in the cell, from low to high.

To evaluate the activity of promoters in the ladder, a set of plasmid based fluorescence reporter constructs was designed. In one example experiment, each promoter in the ladder was cloned in front of eyfp, a gene encoding yellow fluorescent protein in the shuttle vector pK18rep. These plasmids were transformed into C. glutamicum NRRL B-11474 and promoter activity was assessed by measuring the accumulation of YFP protein by spectrometry.

Purified reporter construct plasmids were transformed into C. glutamicum NRRL B-11474 by electroporation (Haynes et al., Journal of General Microbiology, 1990). Transformants were selected on BHI agar plus 25 μg/mL Kanamycin. For each transformation, multiple single colonies were picked and inoculated into individual wells of a 96 mid-well block containing 300 μL of BHI media plus 25 μg/mL Kanamycin. The cells were grown to saturation by incubation for 48 h at 30° C. shaking at 1,000 rpm. After incubation, cultures were centrifuged for 5 min at 3,500 rpm and the media was removed by aspiration. Cells were washed once by resuspension in 300 μL of PBS and centrifugation for 5 min at 3,500 rpm followed by aspiration of the supernatant and a final resuspension in 300 μL of PBS. A 20 μL aliquot of this mixture was transferred to a 96-well full area black clear bottom assay plate containing 180 μL of PBS. The optical density of the cells at 600 nm was measured with the SpectraMax M5 microplate reader and the fluorescence was measured with the TECAN M1000 microplate leader by exciting at 514 nm and measuring emission at 527 nm. For each well a normalized fluorescence activity was calculated by dividing fluorescence by optical density. The parent plasmid pK18rep acted as a negative control. Normalized fluorescence activity was compared between reporter constructs and between biological replicates. A numerical summary of promoter activity is presented in Table 1 below.

TABLE 1
Recombinant C. glutamicum Expressing Yellow Fluorescent Protein
Under the Control of Promoters

| | | | | Standard | 95% | |
|---|---|---|---|---|---|---|
| | | | | Error | Con- | Relative |
| Promoter | No. of | Mean | Standard | of | fidence | Expres- |
| level | Replicates | Activity | Deviation | Mean | Interval | sion |
| 8 | 12 | 114402 | 52987.9 | 15296 | 80735- | 1167 |
| | | | | | 148069 | |
| 7 | 19 | 89243 | 16162.2 | 3708 | 81453- | 911 |
| | | | | | 97033 | |
| 6 | 19 | 44527 | 18110.3 | 4155 | 35798- | 454 |
| | | | | | 53256 | |
| 5 | 10 | 43592 | 3643 | 1152 | 40986- | 445 |
| | | | | | 46198 | |
| 4 | 11 | 11286 | 10459.4 | 3154 | 4260- | 115 |
| | | | | | 18313 | |
| 3 | 19 | 4723 | 1854.3 | 425 | 3829- | 48 |
| | | | | | 5617 | |
| 2 | 18 | 661 | 731.9 | 173 | 297- | 7 |
| | | | | | 1025 | |
| 1 | 14 | 98 | 537.5 | 144 | −212- | 1 |
| | | | | | 409 | |
| No | 20 | −45 | 214.9 | 48 | −145- | |
| promoter | | | | | 56 | |

Promoters levels 1-3 are considered “weak,” promoter levels 4-6 are considered “medium,” and promoter levels 7 and 8 are considered “strong.” In absolute terms, weak promoters here are those with a mean activity less than 6,000; medium promoters have a mean activity of at least 6,000 and no more than 60,000; and strong promoters have a mean activity of more than 60,000. Given that such units are specific both to the species and to the device, relative units have wider applicability. One standard, used in the “Relative Expression” column of Table 1, is that of the weakest promoter in the ladder, assumed to have a mean activity of less than 500 in assays such as those performed here. Weak promoters are those with a relative expression ranging from at least 1 to no more than 60 times the level of the weakest promoter; medium promoters are those with a relative expression ranging from more than 60 to no more than 600 times the level of the weakest promoter; and strong promoters are those with a relative expression of more than 600 times the level of the weakest promoter. Expression levels relative to the characteristics of the cell in which expression takes place are widely applicable across different contexts. For instance, promoters having medium strength can be defined as having at least 20% and no more than 200% of the mean protein expression level within the cell, or as at least 100-fold lower and no more than 10-fold lower than the maximum protein expression level within the cell, where weak and strong promoters are those whose expression level are lower and higher, respectively, than these ranges. Alternatively and more generally, a “medium” promoter could be any that is stronger than the weakest promoter used and weaker than the strongest promoter used.

The metric under consideration in this and other examples is fraction of candidates for improvement, or “hit rate,” which is the fraction of modifications whose measured level of improvement is above a noise threshold in one or more phenotypes of interest. The threshold may be set based on the noise (e.g., root mean squared error) in predicting performance at scale (i.e., larger than small scale) relative to performance at a small, high-throughput scale, and also represents a minimum threshold for what can be considered a substantive improvement in phenotype once confirmed. In embodiments, these cutoffs are 10% above the unmodified parent genome for the productivity model and 3% above parent for the yield model.

Adding a modification into a new strain background is typically done with one of two goals: diversification (search) or consolidation (application). A genetic background strain may be a wild-type strain, or a mutated, engineered strain that contains one or more mutations relative to the wild-type strain. Diversification is the process of attempting as many different modifications as possible in a single strain background, whereas consolidation is the process of applying potentially useful modifications, as identified during the diversification process, to one or more strains backgrounds of interest based on phenotypic performance in the phenotypes of interest (which are productivity and yield in this embodiment), and not necessarily to all that we possibly could. It is useful to consider these two cases separately, since the meaning of a higher or lower fraction of modifications leading to a performance increase above the noise threshold of a phenotype (i.e., hit rate) is different for the two cases. Modifications employed in consolidation are the subset of the best-performing modifications from diversification. A high hit rate in diversification means that improvements are relatively easy to find in a given library, whereas a high hit rate in consolidation means that improvements are consistently valuable in a given library. In other words, during diversification, priority is given to trying as many different modifications as possible in one strain background in order to identify modifications that may be useful in many different backgrounds. A class enriched for hits in diversification means that, in the background used, gene modifications that improved performance were relatively easily found. After potentially useful modifications are identified during diversification, consolidation involves attempting these modifications in multiple backgrounds of interest. Some of these modifications may not prove to be of consistent use in other backgrounds and will not regularly come out as hits. Thus those modifications or classes of modifications that are enriched for hits during consolidation are those that were hits repeatedly in many different strain backgrounds.

As used herein, the term “library” refers to collections of genetic modifications according to the present disclosure. In some embodiments, the libraries of the present invention may manifest as i) a collection of sequence information in a database or other computer file, ii) a collection of genetic constructs encoding for a series of genetic elements, or iii) cell strains comprising said genetic elements. In some embodiments, the libraries of the present disclosure may refer to collections of individual elements (e.g., collections of promoters for PRO swap libraries, or collections of SNPs for SNP swap libraries). In other embodiments, the libraries of the present disclosure may refer to combinations of genetic elements, such as combinations of promoter::genes. In some embodiments, the libraries of the present disclosure may comprise meta data associated with the effects of applying each member of the library in host organisms. For example, a library as used herein can include a collection of promoter::gene sequence combinations, together with the resulting effect of those combinations on one or more phenotypes in a particular species, thus improving the future predictive value of using said combination in future promoter swaps.

Breaking out FIG. 2 by diversification and consolidation yields FIG. 3. FIG. 3 is a modification of FIG. 2, aggregated by library goal—diversification or consolidation. Modifications employed in consolidation are the subset of the best-performing modifications from diversification.

In general, consolidation is the best measure of the value of a library, because success in consolidation results from repeated, consistent utility of a gene modification across multiple backgrounds. In FIG. 3, the differences between promoter strengths are smaller in consolidation than diversification, but the weak promoters still perform most poorly.

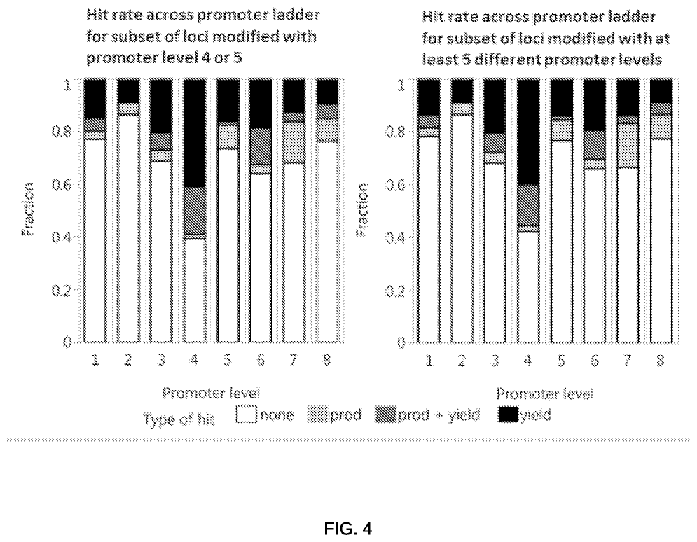

The evidence of medium-strength promoter swaps yielding higher hit rates than strong promoters is particularly demonstrated when the data is limited only to loci that have been subject to medium-strength promoter swaps or loci that have been subjected to more than half (i.e., at least five) of the promoters in the ladder, as shown in FIG. 4. FIG. 4 illustrates subsets of the data from FIG. 2 that are designed to even out the bias in frequency across the different promoter levels.

Thus, the data suggests that medium-strength promoter swaps are more generally useful than strong promoters, which are more useful than weak promoters. Conventional practice in the field is typically to maximize or minimize expression, but such extreme approaches may prove overly taxing to the cell, particularly with respect to modulating essential cellular function.

A number of other modifications are possible beyond promoter swaps. Foreign genes can be inserted or used to replace native genes, single nucleotide polymorphisms (including start codon modifications, such as from ATG to TTG) can be employed, and random mutagenesis via UV, transposons, or other mutagens can also be applied.

Prioritizing Gene Targets Across a Genome

Beyond the nature of what types of modifications should be made, the question of what loci the modifications should be applied to is also addressed in embodiments of the disclosure. Conventionally, metabolic engineers focus their efforts on the metabolic pathway genes. These genes are of obvious importance, and an approach to organizing a genome into shells is start with these genes as “Shell 1.” To define these genes, the collected knowledge of the biosynthesis of the target may be examined to create a list of genes in Shell 1.

In embodiments, an optimization-driven algorithmic method such as flux balance analysis (“FBA”) may be employed to identify genes that will have the maximal impact on diverting the metabolic flux of the organism towards the target product. In such an approach, a genome-scale metabolic model (here, a directed graph of the cellular metabolites connected by gene-catalyzed reactions) of the organism is used to contrast the metabolic phenotype of a strain maximizing the yield of a product in comparison to another phenotype maximizing cellular growth (e.g., base metabolism). The contrast reveals a subset of genes that should be modified (e.g., up-regulated or down-regulated from their expression levels) to alter the base metabolism to a product-maximizing strain. The formal steps of performing the analysis include:
A Linear Programming (LP) optimization problem is formulated to compute, alternatively, the maximum production flux of the target chemical (henceforth the production phenotype) or the maximum cellular growth rate (henceforth the native phenotype) under the assumptions of a metabolic steady state (i.e., the exponential growth phase where there is a net zero rate of accumulation of an intermediate metabolite). The structure of the LP problem is shown below.

$$\begin{matrix}Maximize \\ {v}_{j}\end{matrix} {v}_{target product} or {v}_{cellular growth}$$

$$subject to \text{:}$$

$${\sum }_{j ∈ J}^{ } {S}_{ij} {v}_{j} = 0 , for all metabolites i ( steady - state assumption )$$

$${LB}_{j} \leq {v}_{j} \leq {UB}_{j} , for all reactions j ∈ J ( limits on reaction flux )$$

where Sᵢⱼ is the matrix representation of the topology of the genome-scale metabolic model containing the stoichiometric coefficient of metabolite i taking part in reaction j. The lower LBⱼ and upper UBⱼ limits on the reaction fluxes are imposed based on thermodynamic feasibility that allows reaction to be reversible or restricted to one particular direction. On solving the LP problems, the maximum values for product flux ν_{product}^{max}, and ν_{cellular growth}^{max} cellular growth is saved for the second step.
In the second step, the maximum and minimum feasible flux bound for each reaction j is identified for both the production and native phenotypes. by solving a series of LP problems. All the constraints of the previous problem are imposed, along with an additional constraint restricting minimum flux of the target product and cellular growth to the optimum values ν_{product}^{max} and ν_{cellular growth}^{max} respectively. The structure of the LP problem is shown below.

$$\begin{matrix}Maximize \text{/} Minimize \\ {v}_{j}\end{matrix} {v}_{j} for each reaction j ∈ J$$

$$subject to \text{:}$$

$${\sum }_{j ∈ J}^{ } {S}_{ij} {v}_{j} = 0 , for all metabolites i$$

$${LB}_{j} \leq {v}_{j} \leq {UB}_{j} , for all reactions j ∈ J$$

$${v}_{target product} \geq {v}_{product}^{ma x} or {v}_{cellular growth} \geq {v}_{cellular growth}^{ma x}$$

On solving the LP problems for each of the two phenotypes, the set of feasible flux n production phenotype ranges {LBⱼ^{production phenotype},UBⱼ^{production phenotype}} and {LBⱼ^{native phenotype},UBⱼ^{native phenotype}} are saved.
Contrasting the feasible ranges for each reaction reveals which subset of reactions needs to be up-regulated or down-regulated in its flux capacity to transform the native phenotype towards the production phenotype. In addition, the comparison also provides a quantitative estimate of the level of up/down-regulation required in flux. Gene-reaction maps convey the reaction-level categorization information to identify gene-level manipulations.

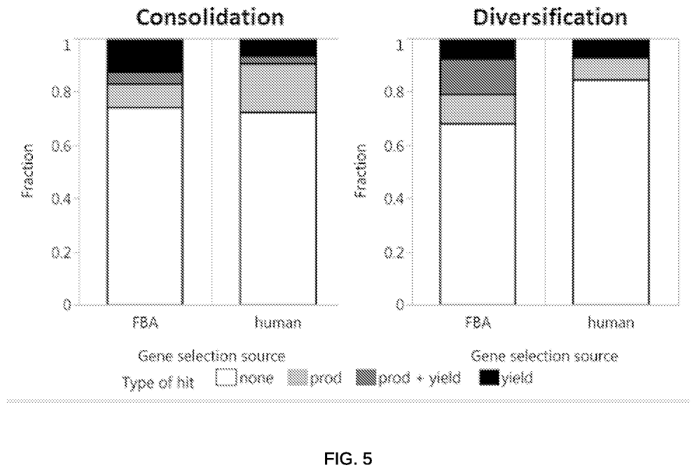

A comparison of the performance of gene modifications determined by these two approaches for the case of optimizing a desired amino acid product yield and productivity in a given microbial strain (e.g., C. glutamicum) is given in FIG. 5.

FIG. 5 illustrates the fraction of modifications whose level of improvement is above a noise threshold for phenotypes of productivity and yield of a target product according to selection by a skilled human or an algorithm (FBA), aggregated by library goal. Modifications employed in consolidation are the subset of the best-performing modifications from diversification obtained during experimentation.

The algorithm recommends more potentially useful changes in the course of diversification, but the rates of valuable changes in consolidation are similar. Another observation is that the algorithm clearly performed better at identifying changes that improve yield or both yield and productivity.

To fully exploit the capacity of an organism for producing a desired product, all its genes should be considered for modification. However, technological limitations still make it difficult to, for example, apply promoter swaps to every gene in a bacterial genome. Thus, embodiments of the disclosure classify and prioritize genes beyond the known on-pathway enzymes for testing. When it comes to genes to target, embodiments of the disclosure determine how to prioritize the genes for modification. One goal of prioritization is to maximize the rate of progress toward a desired performance improvement in the strain of interest.

Another approach to prioritizing genes into shells is via Gene Ontology (GO), according to embodiments of the disclosure. The Gene Ontology classification provides controlled vocabularies of defined terms representing gene product properties. These cover three domains: Cellular Component, the parts of a cell or its extracellular environment; Molecular Function, the elemental activities of a gene product at the molecular level, such as binding or catalysis; and Biological Process, operations or sets of molecular events with a defined beginning and end, pertinent to the functioning of integrated living units: cells, tissues, organs, and organisms.

The GO classification system is structured as a directed acyclic graph where each term has defined relationships to one or more other terms in the same domain, and sometimes to other domains. The GO vocabulary is designed to be species-agnostic, and includes terms applicable to prokaryotes and eukaryotes, and single and multicellular organisms. (See http://geneontology.org/page/ontology-documentation, which is incorporated by reference in its entirety herein).

The Gene Ontology defines the universe of concepts relating to gene functions (GO terms), and how these functions are related to each other (“relations”). It is revised and expanded as biological knowledge accumulates. The GO describes function with respect to three aspects: molecular function (molecular-level activities performed by gene products), cellular component (the locations relative to cellular structures in which a gene product performs a function), and biological process (the larger processes, or “biological programs” accomplished by multiple molecular activities).

Ongoing revisions to the ontology are managed by a team of senior ontology editors with extensive experience in both biology and computational knowledge representation. Ontology updates are made collaboratively between the Gene Ontology Consortium ontology team and scientists who request the updates. Most requests come from scientists making GO annotations (these typically impact only a few terms each), and from domain experts in particular areas of biology (these typically revise an entire “branch” of the ontology comprising many terms and relations).

In an example of GO annotation, the gene product “cytochrome c” can be described by the Molecular Function term “oxidoreductase activity”, the Biological Process term “oxidative phosphorylation”, and the Cellular Component terms “mitochondrial matrix” and “mitochondrial inner membrane”.

Ontologies

Molecular Function

A molecular process that can be carried out by the action of a single macromolecular machine, usually via direct physical interactions with other molecular entities. Function in this sense denotes an action, or activity, that a gene product (or a complex) performs. These actions are described from two distinct but related perspectives: (1) biochemical activity, and (2) role as a component in a larger system/process.

Cellular Component

These terms describe a location, relative to cellular compartments and structures, occupied by a macromolecular machine when it carries out a molecular function. There are two ways in which biologists describe locations of gene products: (1) relative to cellular structures (e.g., cytoplasmic side of plasma membrane) or compartments (e.g., mitochondrion), and (2) the stable macromolecular complexes of which they are parts (e.g., the ribosome). Unlike the other aspects of GO, cellular component concepts refer not to processes but rather a cellular anatomy.

Biological Process

A biological process represents a specific objective that the organism is genetically programmed to achieve. Biological processes are often described by their outcome or ending state, e.g., the biological process of cell division results in the creation of two daughter cells (a divided cell) from a single parent cell. A biological process is accomplished by a particular set of molecular functions carried out by specific gene products (or macromolecular complexes), often in a highly regulated manner and in a particular temporal sequence.

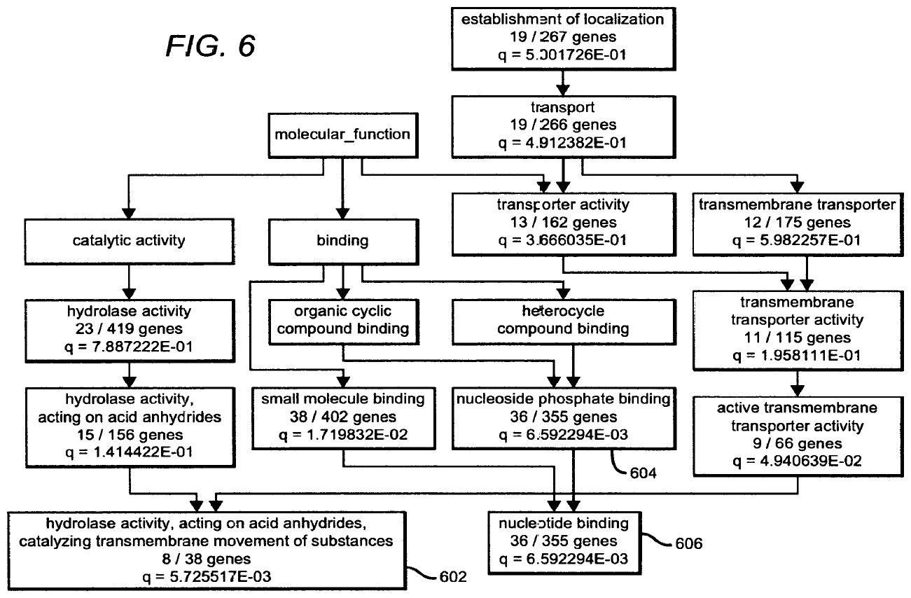

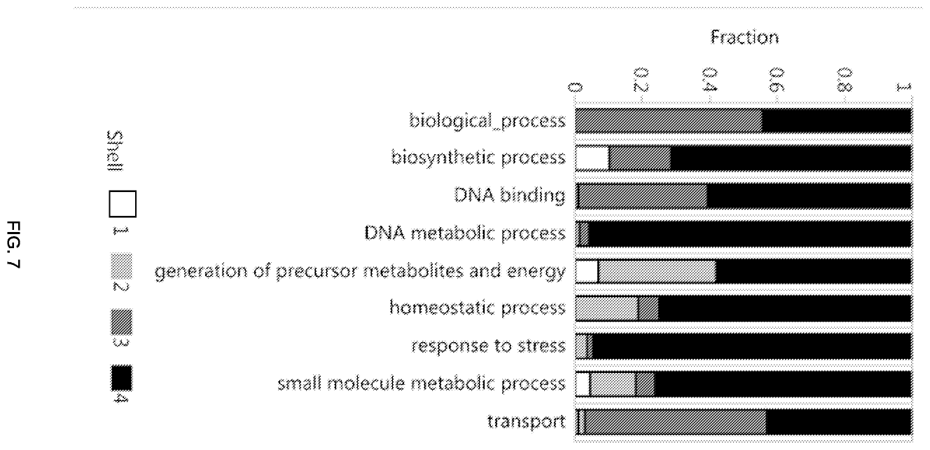

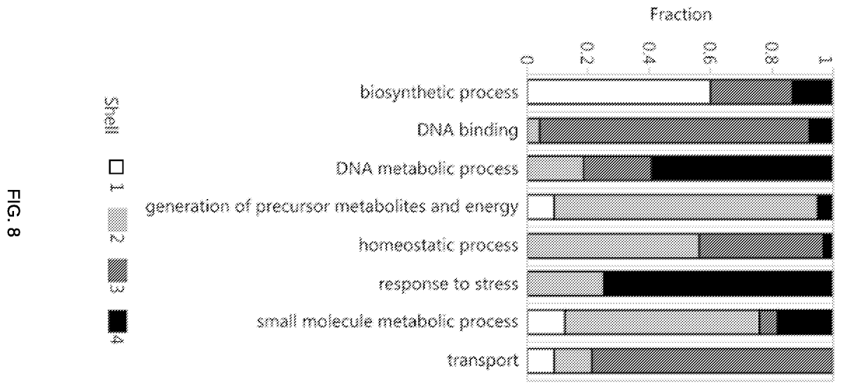

FIG. 6 illustrates an example of a subgraph from the Gene Ontology, with gene classes 602, 604 and 606 enriched for improved yield. In this grouping, gene sets are associated with specific terms in the ontology (and all ancestral terms). All terms (other than the root terms representing each namespace, above) have a sub-class relationship to another term.

The following is an example of a GO term taken from the OBO format file.
id: GO:0016049
name: cell growth
namespace: biological process
def: “The process in which a cell irreversibly increases in size over time by accretion and biosynthetic production of matter similar to that already present.” [GOC:ai]
subset: goslim_generic
subset: goslim_plant
subset: gosubset_prok
synonym: “cell expansion” RELATED [ ]
synonym: “cellular growth” EXACT [ ]
synonym: “growth of cell” EXACT [ ]
is_a: GO:0009987 ! cellular process
is_a: GO:0040007 ! growth
relationship: part_of GO:0008361 ! regulation of cell size
   http://geneontology.org/page/ontology-structure

Gene ontologies can be “rolled up” into various levels of abstraction and aggregation using GO Slims, which are subsets of GO terms that give a more general overview of gene classification (see http://geneontology.org/page/go-slim-and-subset-guide). In this case, to “roll up” a GO term means to start from classification of genes according to a specific GO term and move “up” the graph from that more specific term to classify those genes under a more general GO term of which the specific term is a subset. The “roll up” process can continue from there, moving from the general GO term to an even more general GO term that incorporates this. This process continues until one or more GO terms that are contained within a much smaller list of general GO terms is reached. In this way, each specific GO term is converted into a more general GO term contained within the limited list of GO terms within the GO Slim ontology file. The use of GO Slims is of most potential use for prioritizing a genome into shells.

Algorithmically defining a GO SLIM mapping may include methods such as rolling all GO terms up three levels, or doing an iterative rollup until hitting a “sweet spot” in terms of number of total GO terms, or number of genes assigned per given GO term. Embodiments of the disclosure may define the “sweet spot” approach algorithmically so that GO terms are stepwise rolled up until all pools of GO Slims reach a defined size, or the pool of unique GO terms has been reduced by a specific amount. These approaches have the advantage of being easily extensible to many other cases.

TABLE 2

| | | | Productivity |
|---|---|---|---|
| GO ID | Name | Yield enriched? | enriched? |
| GO: 0003677 | DNA binding | Yes | Yes |
| GO: 0006810 | transport | Yes | No |
| GO: 0006091 | generation of precursor | Yes | No |
| | metabolites and energy | | |
| GO: 0042592 | homeostatic process | Yes | Yes |
| GO: 0044281 | small molecule metabolic | Yes | Yes |
| | process | | |
| GO: 0008150 | biological_process | Yes | Yes |
| GO: 0009058 | biosynthetic process | Yes | Yes |
| GO: 0006259 | DNA metabolic process | No | Yes |
| GO: 0006950 | response to stress | No | Yes |

Table 2 shows GO Slim terms enriched for a desired amino acid yield and productivity in a given microbial strain based on experimentation. For each GO term, the number of genes resulting in a yield or productivity improvement above a preset threshold were compared to the number that would be expected to be seen by chance. This table is for consolidation and diversification combined, and is dominated by diversification experiments.

Once a gene classification scheme has been decided upon, the next step is to explain the structure of experimental effect in terms of the classification; i.e., determine which subclasses are most useful for improving the target phenotype, to guide subsequent rounds of modification, or to apply analogously to another target and/or organism. Statistical or machine learning approaches may be employed to identify these subclasses.

Among statistical approaches, Gene Set Enrichment Analysis (“GSEA”) may be employed in embodiments of the disclosure. (See GSEA; Subramanian A., et al. “Gene set enrichment analysis: A knowledge-based approach for interpreting genome-wide expression profiles,” PNAS, 2005, incorporated by reference in its entirety herein.) GSEA attempts to identify a subset of gene classes within an ontology that are overrepresented among a set of candidate genes. This analysis typically provides two types of output: an enrichment score ES indicating the degree of enrichment, and a p-value indicating the significance of the result. Statistical methods may be employed to correct for multi-hypothesis testing.

While the completion of the Human Genome Project gifted researchers with an enormous amount of new information, it also left them with the problem of how to interpret and analyze the incredible amount of resulting data. To seek out genes associated with diseases, researchers utilized DNA microarrays, which measure the amount of gene expression in different cells. Researchers performed these microarrays on thousands of different genes, and compare the results of two different cell categories, e.g. normal cells versus cancerous cells. However, this method of comparison is not sensitive enough to detect the subtle differences between the expression of individual genes, because diseases typically involve entire groups of genes. Multiple genes are linked to a single biological pathway, and so it is the additive change in expression within gene sets that leads to the difference in phenotypic expression. Gene Set Enrichment Analysis focuses on the changes of expression in groups of genes, and by doing so, this method resolves the problem of the undetectable, small changes in the expression of single genes.

Gene set enrichment analysis uses a priori gene sets that have been grouped together by their involvement in the same biological pathway, or by proximal location on a chromosome—all of which may serve as modification features. In embodiments of the disclosure, a database of these predefined sets may be found at The Molecular Signatures Database (MSigDB). In GSEA, DNA microarrays, or now RNA-Seq (whole transcriptome shotgun sequencing) may be performed and compared between two cell categories, but instead of focusing on individual genes in a long list, the focus is on a gene set. Researchers analyze whether the majority of genes in the set fall in the extremes of this list: the top and bottom of the list correspond to the largest differences in expression between the two cell types. If the gene set falls at either the top (over-expressed) or bottom (under-expressed), it is thought to be related to the phenotypic differences.

Genome-wide association studies may be employed, for example, in comparisons between healthy and disease genotypes to try to find SNPs that are overrepresented in the disease genomes, and might be associated with that condition. Before GSEA, the accuracy of genome-wide SNP association studies was severely limited by a high number of false positives. The GSEA-SNP method is based on the theory that the SNPs contributing to a disease tend to be grouped in a set of genes that are all involved in the same biological pathway. This application of GSEA not only aids in the discovery of disease-associated SNPs, but helps illuminate the corresponding pathways and mechanisms of the diseases.

Alternatively, embodiments of the disclosure may apply machine learning (“ML”) techniques to learn the relationship between the given classes (features) of an ontology and observed outcomes. In this framework, embodiments may use standard ML models, e.g. Decision Trees, to determine feature importance. Because of the hierarchical nature of ontology classes, features are often correlated or redundant, which can lead to ambiguous model fitting and feature inspection. To address this issue, dimensional reduction may be performed on input features via principal component analysis. Alternatively, feature trimming may be performed based on information gained from child to parent ontology classes.

In general, machine learning may be described as the optimization of performance criteria, e.g., parameters, techniques or other features, in the performance of an informational task (such as classification or regression) using a limited number of examples of labeled data, and then performing the same task on unknown data. In supervised machine learning such as an approach employing linear regression, the machine (e.g., a computing device) learns, for example, by identifying patterns, categories, statistical relationships, or other attributes, exhibited by training data. The result of the learning is then used to predict whether new data will exhibit the same patterns, categories, statistical relationships or other attributes.

Embodiments of the disclosure may employ other supervised machine learning techniques when training data is available. In the absence of training data, embodiments may employ unsupervised machine learning. Alternatively, embodiments may employ semi-supervised machine learning, using a small amount of labeled data and a large amount of unlabeled data. Embodiments may also employ feature selection to select the subset of the most relevant features to optimize performance of the machine learning model. Depending upon the type of machine learning approach selected, as alternatives or in addition to linear regression, embodiments may employ for example, logistic regression, neural networks, support vector machines (SVMs), decision trees, hidden Markov models, Bayesian networks, Gram Schmidt, reinforcement-based learning, cluster-based learning including hierarchical clustering, genetic algorithms, and any other suitable learning machines known in the art. In particular, embodiments may employ logistic regression to provide probabilities of classification (e.g., classification of genes into different functional groups) along with the classifications themselves. See, e.g., Shevade, A simple and efficient algorithm for gene selection using sparse logistic regression, Bioinformatics, Vol. 19, No. 17 2003, pp. 2246-2253, Leng, et al., Classification using functional data analysis for temporal gene expression data, Bioinformatics, Vol. 22, No. 1, Oxford University Press (2006), pp. 68-76, all of which are incorporated by reference in their entirety herein.

Embodiments may employ graphics processing unit (GPU) accelerated architectures that have found increasing popularity in performing machine learning tasks, particularly in the form known as deep neural networks (DNN). Embodiments of the disclosure may employ GPU-based machine learning, such as that described in GPU-Based Deep Learning Inference: A Performance and Power Analysis, NVidia Whitepaper, November 2015, Dahl, et al., Multi-task Neural Networks for QSAR Predictions, Dept. of Computer Science, Univ. of Toronto, June 2014 (arXiv:1406.1231 [stat.ML]), all of which are incorporated by reference in their entirety herein. Machine learning techniques applicable to embodiments of the disclosure may also be found in, among other references, Libbrecht, et al., Machine learning applications in genetics and genomics, Nature Reviews: Genetics, Vol. 16, June 2015, Kashyap, et al., Big Data Analytics in Bioinformatics: A Machine Learning Perspective, Journal of Latex Class Files, Vol. 13, No. 9, September 2014 (arXiv:1506.05101), Prompramote, et al., Machine Learning in Bioinformatics, Chapter 5 of Bioinformatics Technologies, pp. 117-153, Springer Berlin Heidelberg 2005, all of which are incorporated by reference in their entirety herein.

GSEA for Strain Optimization—Learning New Ontological Classes

In embodiments, GSEA may be used in the context of a strain optimization problem to learn novel ontological classes based on a set of historical data, and to use those learned classes to predict new candidate changes that are likely to improve performance. GSEA may be used to determine target genes, and it may also be combined with other information (such as knowledge of optimum promoter strength levels) to select the modifications to be performed.

Embodiments of the disclosure make predictions for untested genes. For instance, the present strain optimization project made use of human experts-to prioritize the genome into four shells, consisting of 26, 81, 415, and 2107 genes. Currently the first three shells are complete, and approximately one half of the last (fourth) shell has been completed. The last shell represents the remaining ˜80% of the genome that was not obvious to a human expert as important to optimizing the target yield and productivity phenotypes. However, progress to date through the last shell by the assignee of the invention has resulted in numerous useful phenotypic improvements, and thus better prioritizing these genes is a priority. “Progress” here refers to the fraction of Shell 4 genes that have actually had modifications applied to them. The correspondence of enriched GO slims from Table 2 to the human-defined shells is given in FIG. 7.

FIG. 7 illustrates a breakdown of genes in the enriched GO Slims of Table 2, by correspondence to human prioritized shells of all genes in a strain genome of interest.

Under one approach, embodiments of the disclosure prioritize the last shell by focusing on those GO slims that are highly represented in the last shell. Examples from FIG. 7 include “DNA binding,” “DNA metabolic processes,” and “response to stress.” Thus, embodiments of the disclosure prioritize the application of gene modifications to genes within those GO slims before performing gene modifications on genes in other GO slims.

Embodiments of the disclosure may also consider where useful modifications have previously come from. For example, FIG. 8 shows which human-designed shells include the modifications to date judged to be “hits” (candidate phenotypic improvements above noise) that correspond to the GO slims shown in FIG. 8.

FIG. 8 illustrates the breakdown of the subset of genes in enriched GO slims whose modification via promoter swap has been demonstrated to improve a desired phenotype, by correspondence to human prioritized shells of all genes in the exemplary strain genome of interest.

Embodiments of the disclosure consider those GO slims that have led to useful improvements in Shell 4 as likely to continue to produce useful improvements. Examples from FIG. 8 include “DNA metabolic process” and “response to stress.” These two GO slims represent 91 genes, 46 of which have previously been targets of modification; the remaining 45 genes can thus be considered high priority targets for the next phase.

Embodiments of the disclosure employ machine learning approaches to evaluate the utility of the above approach retrospectively. An example process is:
Split historical data into training and test sets
Compute per-class enrichment probability using the training data set, e.g., using GSEA.
Predict enrichment probability for all gene class instances not present in the training set (i.e., the test data set).
Compare predicted vs. observed per-class enrichment probabilities with respect to the test data set.
Tune any hyperparameters, e.g., decision tree parameters in an ML algorithm, as needed.

Online Learning

In consideration of the above, embodiments of the disclosure may initially prioritize genes as candidates for modification, categorized into shells, in the following descending order:

1. Genes identified as targets by FBA or another metabolic model, or combination thereof (including metabolism maps and literature consulted by expert humans)
2. GO slims identified as useful in previous genome-wide metabolic optimization projects efforts that seem applicable (e.g., DNA metabolism, gene regulation, stress response), as well as any GO slims judged likely to be important by expert humans
3. Other genes

After the initial shells have been completed and some progress has been made in the last shell, embodiments of the disclosure may iteratively perform an automated GSEA or other analysis, and re-prioritize the remaining final-shell genes. In embodiments, the prioritization engine may rely on experimental outcomes to force the weighting of certain features in the prediction algorithm. For example, weights may be assigned to the following gene sets in the following order from heaviest to lightest weighting:

1. Genes in enriched GO slims that have previously generated useful improvements from among final-shell genes
2. Genes in enriched GO slims that are well-represented in the final shell
3. Other genes in enriched GO slims
4. Other genes

In embodiments, medium-strength promoter swaps may be attempted first, followed by strong promoters, with weak promoters receiving the lowest priority. Note also that in cases where a gene belongs to multiple classes, either because classes are overlapping or because multiple classification systems have been employed, a weighted predicted performance can be assigned for each gene based on the combination of predicted performance pertaining to each of the classes to which it belongs. Weighting the predicted performance would affect the corresponding prioritization accordingly. In the simplest case, the mean class-based predicted performance of each gene could be used. Another example would be a mean class-based predicted performance weighted according to the size or known utility of each relevant class.

As new sets of gene modifications are predicted, applied and tested, data can be collected about which classes of modifications are most useful, which can then be fed back in “online” fashion to prioritizing the next round of modifications. In more algorithmic terms, GSEA models can be iteratively updated via Thompson sampling to efficiently learn the most relevant (i.e., hit-enriched) ontological classes, as described below. This technique adjusts the proportional sampling of classes based upon past per-class success (e.g., performance improvement hits).
Assume an ontology O of classes Cᵢ and a mapping between ontology classes and genes. Assume per-cycle strain-build capacity N (e.g., number of strains built per cycle)
Initialize
   j=0. Here j is the main while loop counter.
   jₘₐₓ the maximum number of runs to perform.
   prior ontology class expected enrichment rates Pⱼ(Cᵢ), where j is the iteration and i is the index identifying the ontology class, based upon prior knowledge from experimental data, other techniques such as the FBA or other metabolic models, or other techniques discussed above with respect to initial prioritization.
   strain performance goal y_{goal}=0, and current parent strain performance yⱼₖ=0, as the baseline, k represents the k^{th} strain built in round j.
While max(yⱼₖ)<y_{goal} or j<jₘₐₓ
   Sample N genes gₖ at random from ontology classes Cᵢ in proportion to Pⱼ(Cᵢ). That is, perform Thompson Sampling from the ontology classes. Sampling may be performed with or without replacement. One skilled in the art may recognize that other learning policies such as the Knowledge Gradient policy may alternatively be employed.
   Apply one of the gene perturbation techniques, such as promoter swapping, targeting genes gₖ identified in the previous step. This results in new strains sⱼₖ
   Measure the phenotypic performance of the new strains: yⱼₖ=f(sⱼₖ)
   Determine updated ontology class enrichment rate Pⱼ₊₁(Cᵢ) based on new measurement results using GSEA or other techniques described above.
   Increment j=j+1

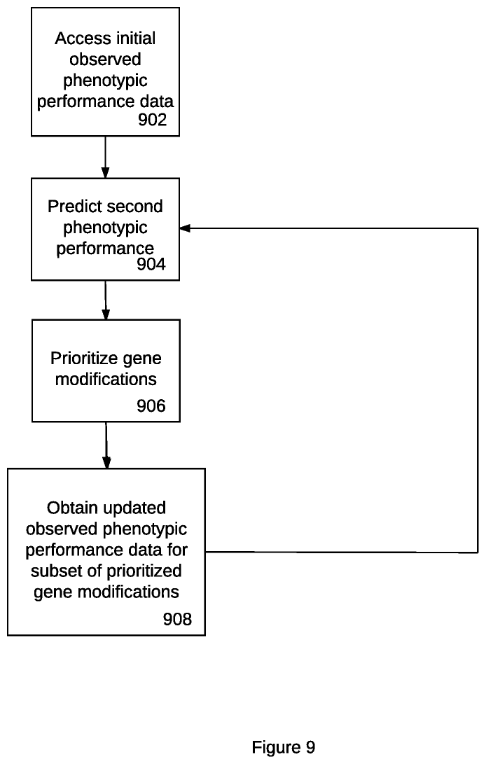

According to embodiments, referring to FIG. 9, the prioritization engine accesses first phenotypic performance data based at least in part upon first gene modifications made to a first set of genes in at least one microbial strain (902); predicts second, predicted phenotypic performance of second gene modifications based at least in part upon the first phenotypic performance data and at least one modification feature that is common to the first gene modifications and the second gene modifications (904); and prioritizes the second gene modifications to be applied to a second set of genes based at least in part upon the second phenotypic performance (906). Based at least in part upon the prioritizing, second gene modifications may be applied to genes within at least one microbial strain. A modification feature is a parameter considered to be of possible utility in predictive modeling, e.g., machine learning. Modification features may be expressed as categorical features (e.g., a type), continuous (e.g., a number), or ordinal features (e.g., discrete groups, such as better or worse).

The prioritization engine may iteratively update prioritization of subsets of the second gene modifications to be applied to subsets of genes within the second set of genes based upon phenotypic performance data observed from iterative application of one or more gene modifications of the second gene modifications to genes within the second set of genes.

In embodiments, the prioritization engine may obtain updated first, observed phenotypic performance data based at least in part upon application of one or more gene modifications of the second gene modifications to genes within the second set of genes (908), and predict updated second phenotypic performance of a subset of the second gene modifications based at least in part upon the updated first phenotypic performance data (904). The prioritization engine may then update the prioritization of the subset of the second gene modifications to be applied to a subset of genes within the second set of genes based at least in part upon the updated second phenotypic performance (906). Note that the application of one or more gene modifications of the second gene modifications to genes within the second set of genes effectively moves those modified genes from within the second set of genes to the first set of genes, for which performance data may now be obtained, according to embodiments of the disclosure. According to embodiments of the disclosure, any combination of the embodiments described herein may be used to produce microbial strains using the prioritized genetic modifications. According to embodiments of the disclosure, a microbial strain is produced to comprise a first gene modification applied to a gene in the first set of genes. According to embodiments, such a microbial strain may further comprise a second gene modification that is prioritized above a threshold prioritization and applied to at least one gene in the second set of genes, wherein the applied gene modification is prioritized higher in response to the prioritization being based on the predicted updated second phenotypic performance than in response to being based on the predicted second phenotypic performance.

According to embodiments of the disclosure, the gene modifications and the at least one modification feature may relate to the genes to be modified or to the types of modifications to be made to those genes. For example, the at least one modification feature may include class, including ontological class, such as class related to GO classification, or the type of modification, such as a promoter swap (e.g., a promoter modification, including insertion, deletion, or replacement of a promoter), or a SNP (single nucleotide polymorphism) swap (e.g., a single base pair modification, including insertion, deletion or replacement of a single base pair).

The modification feature may be related to the strength of the promoter, such as weak, strong, or medium strength. Experiments by the inventors have shown instances where medium strength promoters generated a greater likelihood of performance (e.g., yield, productivity) improvement by the microbial strain than did weak or strong promoters. Thus, the prioritization engine may weight medium-strength promoters more heavily than strong or weak promoters into the predicted phenotypic performance. In embodiments of the disclosure, the prioritization engine may weight weak promoters less heavily than strong and medium-strength promoters.

In general, the prioritization engine may weight known beneficial effects more heavily into the predicted phenotypic performance than lesser effects. Conversely, in embodiments the prioritization engine may assign low weighting to known negative or less beneficial effects in the predicted phenotypic performance than more beneficial effects. As another example, in embodiments predicting second phenotypic performance of second gene modifications is based at least in part upon at least one modification feature including modifications of one or more types (e.g., promoter swap, SNP swap) to at least two genes in a strain. In this manner, the method accounts for epistatic effects arising from the phenotypic effects of making two or more gene modifications to the same strain. In such embodiments, predicting may more heavily weight, into the predicted phenotypic performance, modifications of one or more types that yield positive epistatic effects.

In embodiments, the at least one modification feature includes different levels of abstraction within a gene ontology classification. In embodiments, the at least one modification feature includes classification based upon metabolic network. In embodiments, the second set of genes includes no genes within the first set of genes. In embodiments, genes within the second set of genes are each a member of multiple classes, and a composite performance prediction for a given gene can be generated from the combination of predictions applying to each class to which it belongs. In embodiments, genes within the second set of genes share membership in at least one common class, and such genes are all assigned the same predicted performance if the common class is the only class to which each gene belongs. In embodiments, genes within the second set of genes may each be a member of only a single class. In embodiments, genes in the first and second sets may share class membership with each other and such genes may each belong to multiple classes.

In embodiments, the at least one modification feature includes first ontological classes from a first classification system and second ontological classes from a second classification system. If, for example, a gene is a member of multiple classes from different classification systems (e.g., GO, KEGG, gene or gene-product sequence similarity, protein domain) and those classes have been observed or predicted to yield performance improvements, then the the prioritization engine may favorably weight the predicted phenotypic performance of that gene as a candidate for modification (thereby increasing its chance of being assigned a high priority), according to embodiments of the disclosure.

In embodiments, the at least one modification feature includes a characteristic of the product produced by at least one microbial strain. For example, the characteristic of the product may be related to the same metabolic pathway or ontological class. If the first set or a gene from the first set are associated with a performance improvement, then it is likely that a gene from the second set along the same metabolic pathway or within the same ontological class would also give rise to a performance improvement. Thus, the the prioritization engine may favorably weight the predicted phenotypic performance of that gene as a candidate for modification (thereby increasing its chance of being assigned a high priority), according to embodiments of the disclosure.

Alternatively, if multiple strain-product combinations are used as modification features of phenotypic performance data, characteristics of the product may be used to weight the relevance of data relating to an input strain-product combination to the target strain-product combination. Inputs that share more characteristics with the target product are more likely to yield useful predictions. In embodiments, those product characteristics may include number of constituent atoms, structure, atomic content, being produced from closely related (either by content or distance to nearest common precursor) metabolic pathways, or the like, with the first product.

In embodiments, the prioritization engine may employ machine learning using genes from the first set of genes as a training set in a machine learning predictive model to predict the second phenotypic performance of the second gene modifications.

In embodiments, the prioritization engine may predict second phenotypic performance by predicting per-class enrichment probabilities for the second gene modifications based at least in part upon the first, observed phenotypic performance data, and prioritizing the second, predicted gene modifications based at least in part upon a ranking of the predicted per-class enrichment probabilities. In embodiments of the disclosure, the prioritization engine may prioritize at least one candidate gene for testing within a class if the predicted enrichment for the class exceeds a threshold enrichment.

In embodiments, the at least one modification feature relates to a characteristic of microbial strain. Such features may include phylogenetic or taxonomic features, including genomic sequence similarity, domain (Archaea, Bacteria, or Eukarya), Gram positive or negative (for the bacteria), genus, species, and the like; ecological and physiological features, including features of the native environment (e.g., pH, temperature, salinity, pressure), metabolic features (e.g., preferred growth substrates, possible growth substrates, waste products), and the like; or other features. For example, if a modification to a set of genes in a first strain provides a performance improvement, then it is likely that a similar modification to a similar set of genes in a similar, second strain would also give rise to a performance improvement. “Similar set of genes” here may be defined as, e.g., genes belonging to the same gene ontology class, belonging to a metabolic pathway having the same product, sequence similarity, similarity in expression profile or regulation, or the like. “Similar” strains may be characterized by phylogentic similarity, similarlity in genetic lineage; whether the strains are prokaryotic or eukaryotic, consume similar feedstock, produce the similar metabolites, or are similar in other modification features. Thus, the method may favorably weight the predicted phenotypic performance of genes within that similar set in the second strain as candidates for modification by the same or a similar modification, according to embodiments of the disclosure.

In embodiments, the second set of genes resides within at least one microbial strain different from the at least one microbial strain in which the first set of genes resides. In those embodiments and others, the first phenotypic performance data may relate to at one or more characteristics of a first product produced by the at least one microbial strain, and the second, predicted phenotypic performance may relate to one or more characteristics of a second product that is different from the first product, and produced by the same strain or another strain sharing common features. In embodiments, the second product may share common features, such as number of constituent atoms, structure, atomic content, being produced from closely related (either by content or distance to nearest common precursor) metabolic pathways, or the like, with the first product.

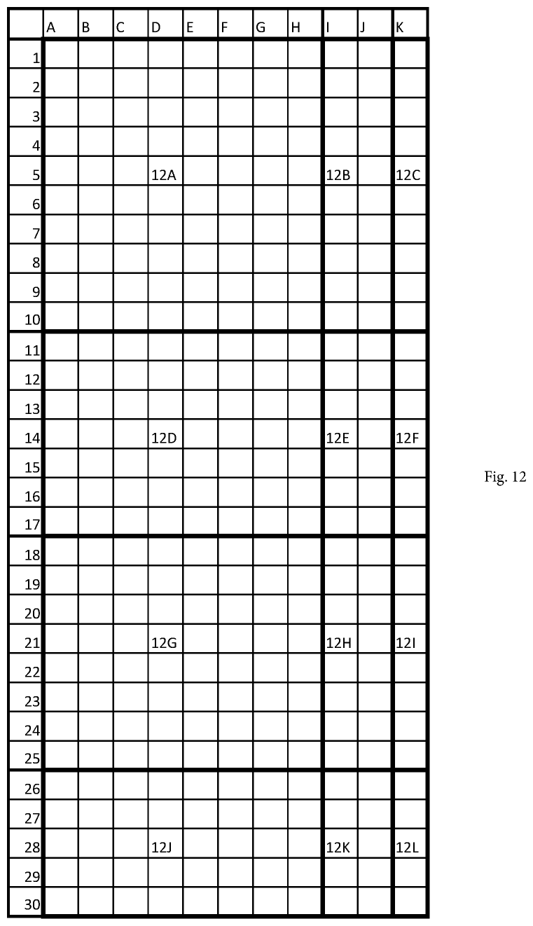

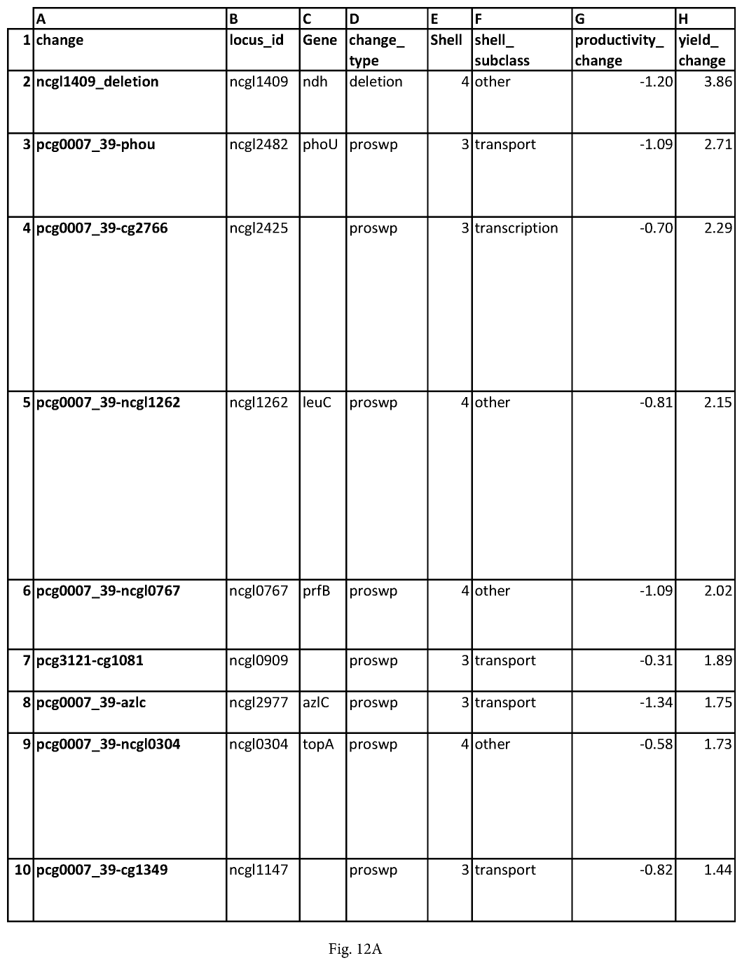

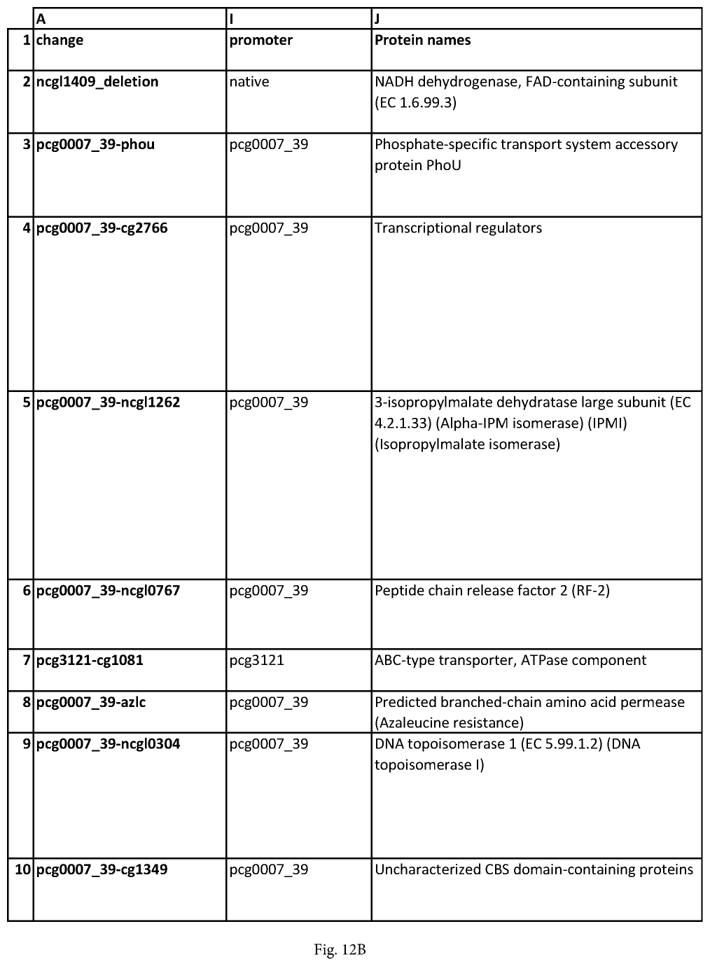

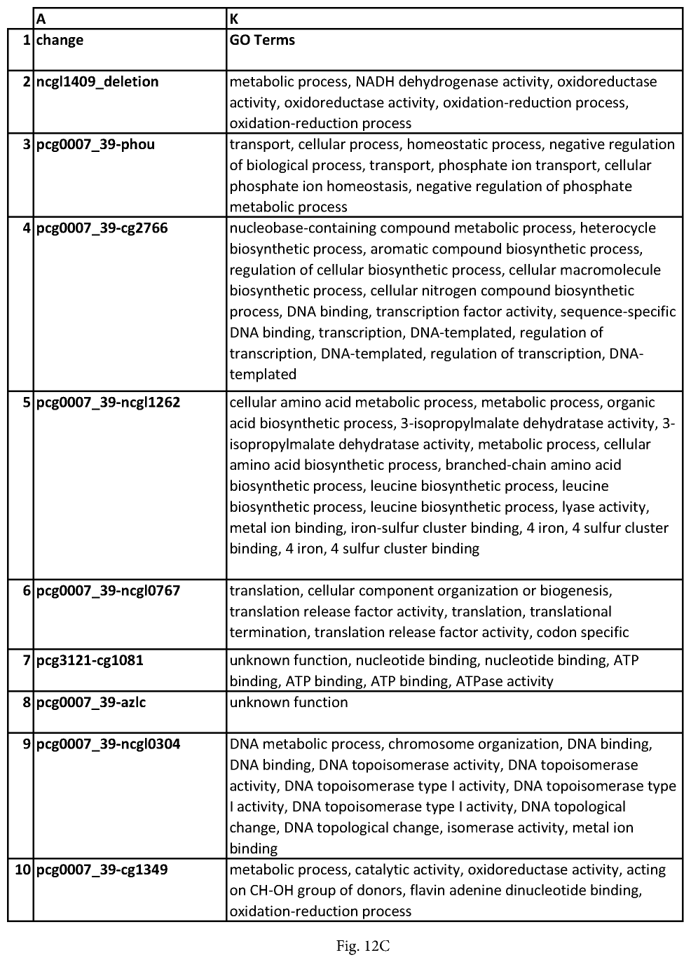

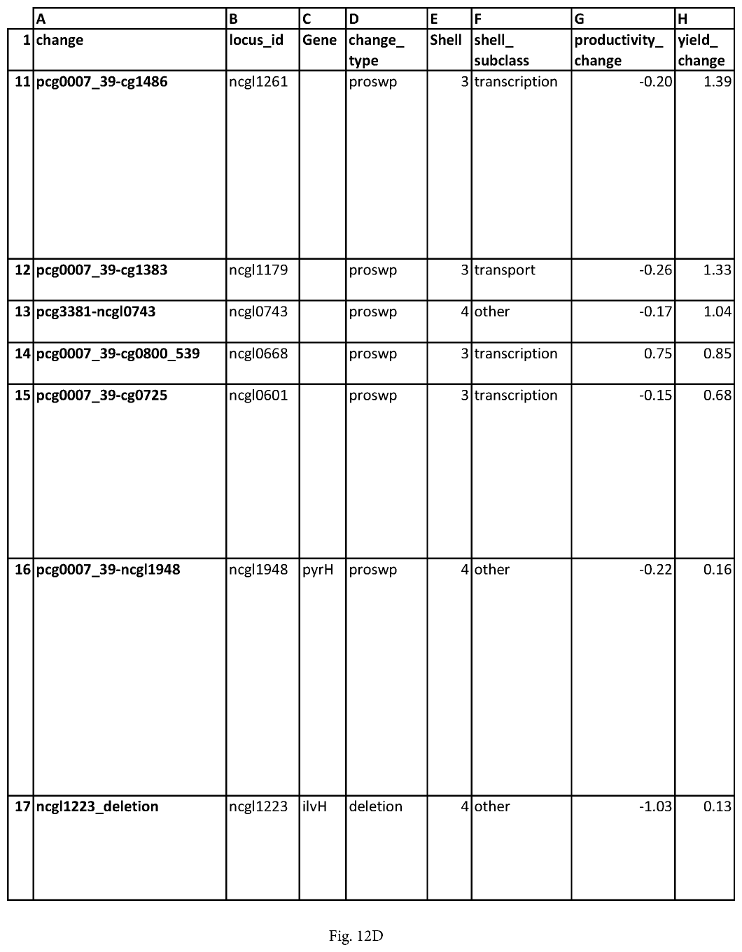

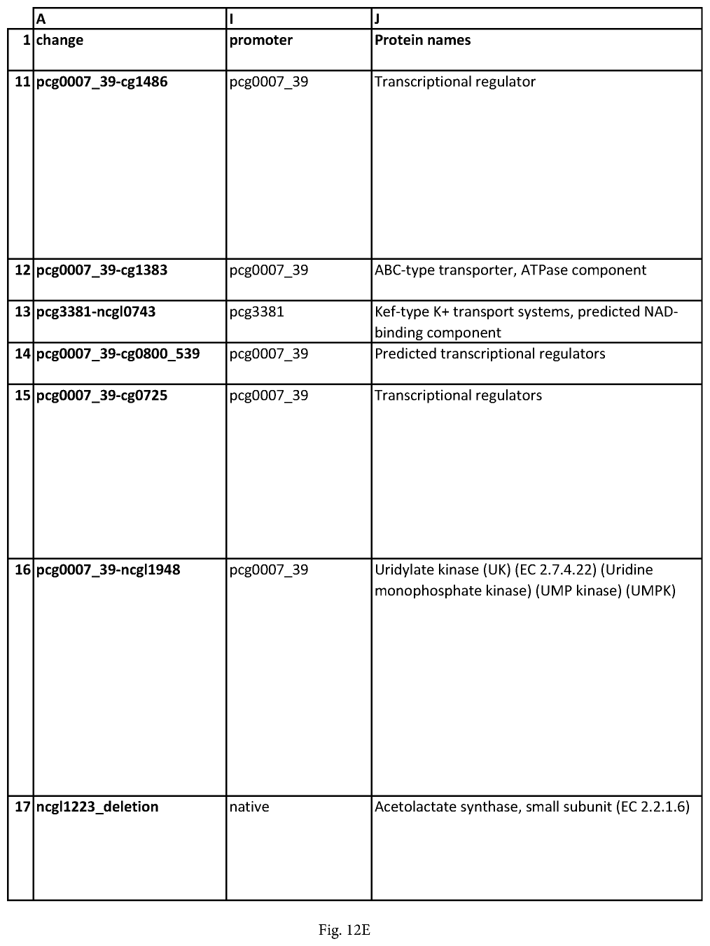

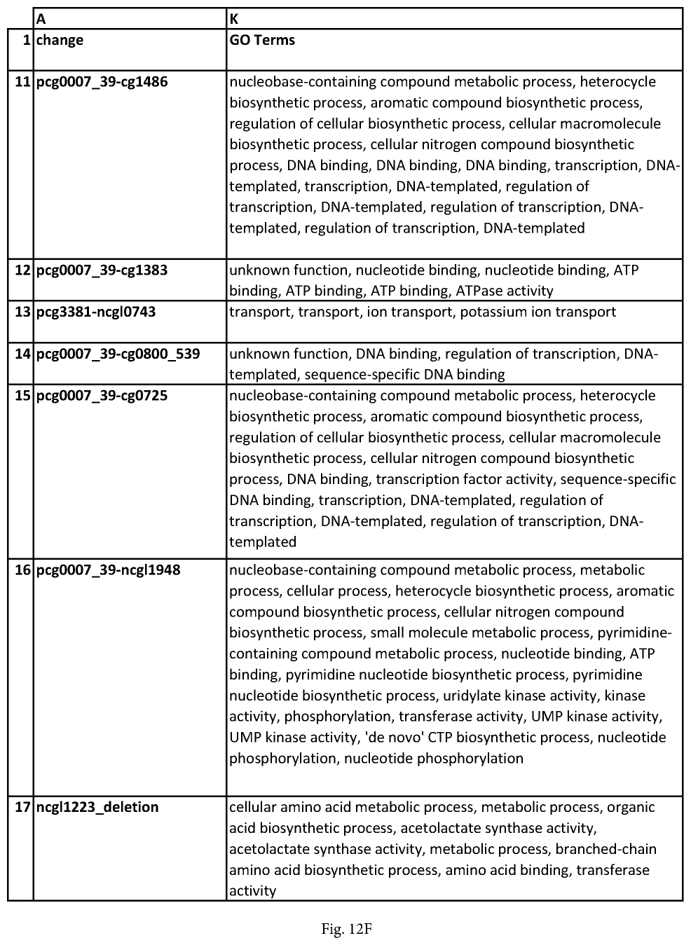

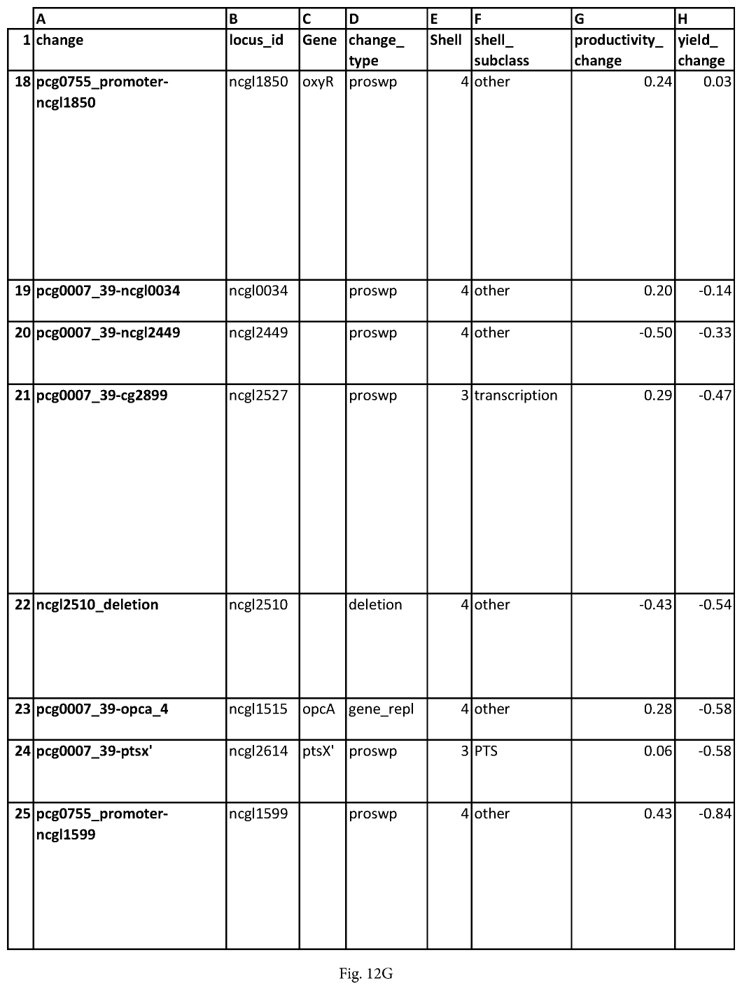

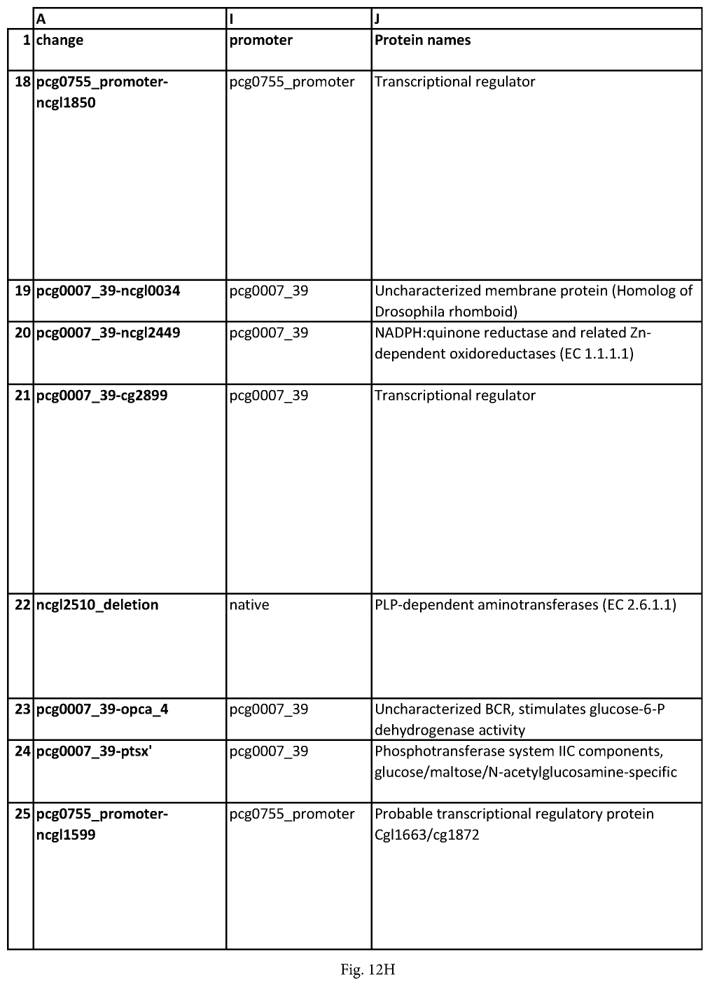

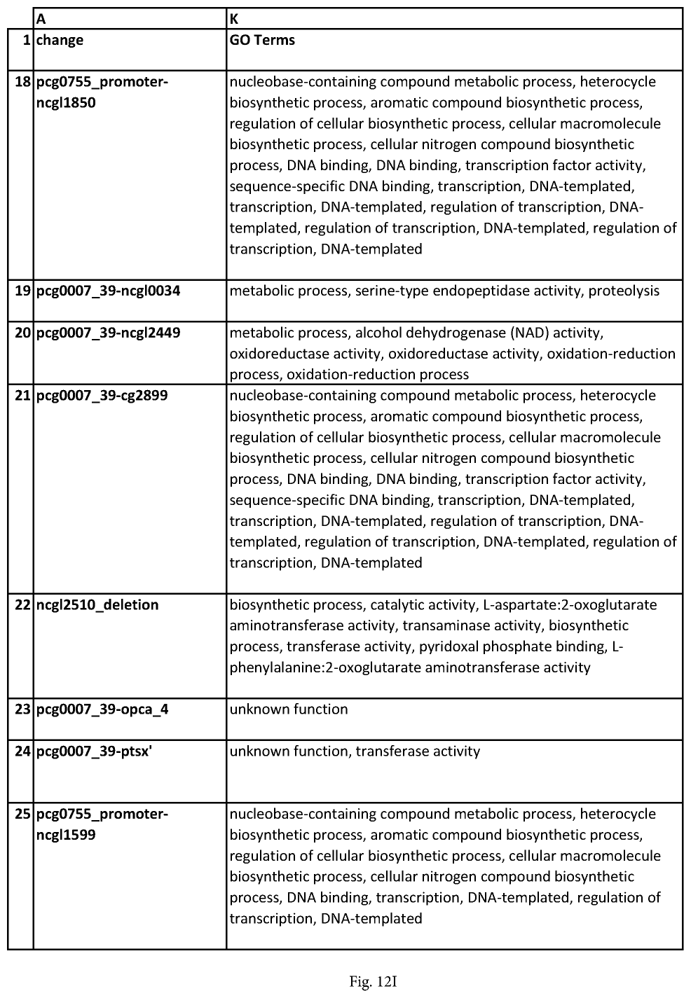

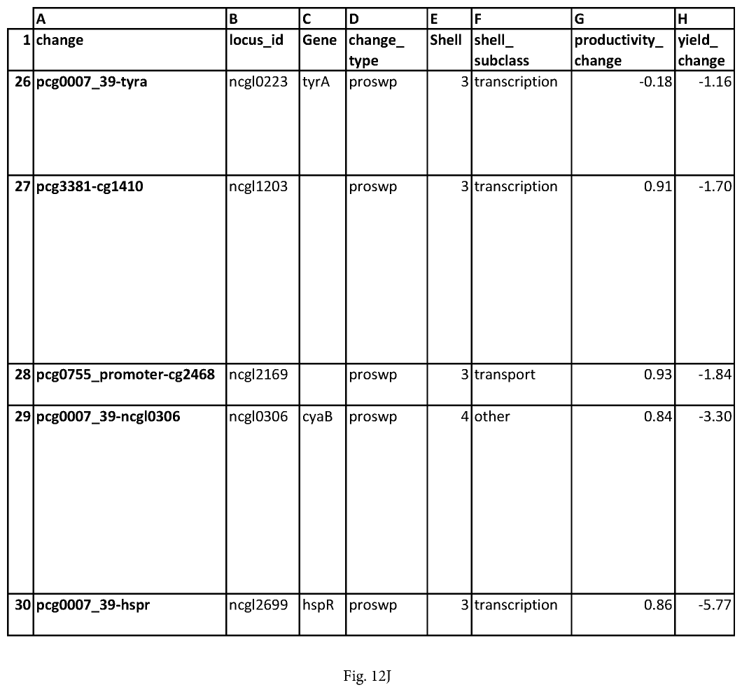

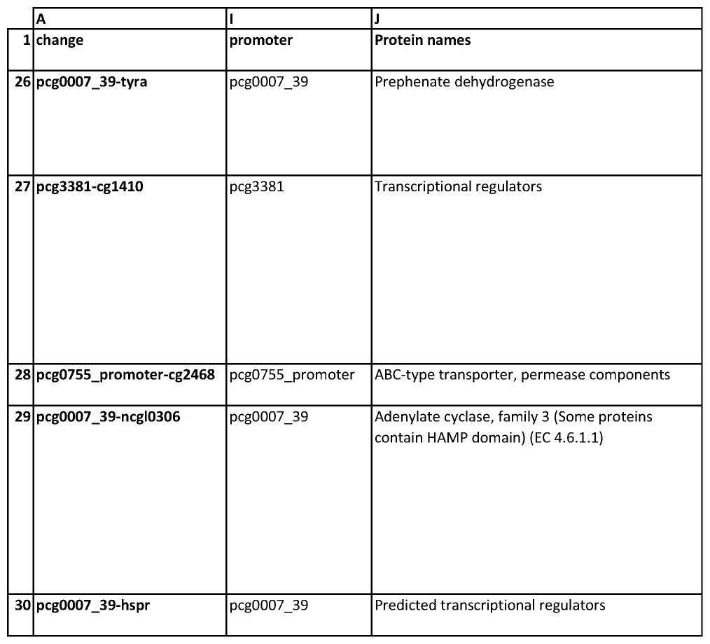

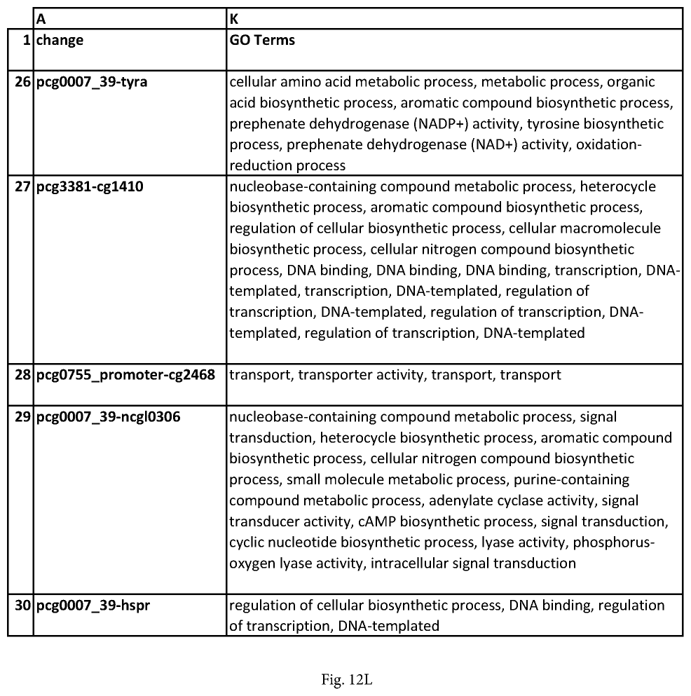

FIG. 12 is a diagram that serves as a guide to the layout of the table segments of FIGS. 12A-12L. FIGS. 12A-12L together form a table of experimental data illustrating attributes involved in the production of particular amino acid in a particular microbial host organism. (The table can also be pieced together without the guide of FIG. 12 by reference to the row and column numbers in each of FIGS. 12A-12L.) Reading across the column headings (identified in parentheses) for any row, one can see the change (A) (identified by a change identifier) that affects the host gene (C), under standard nomenclature (also identified by locus_id (B) under ngcl nomenclature referenced in M. Ikeda, et al., The Corynebacterium glutamicum genome: features and impacts on biotechnological processes, Appl Microbiol Biotechnol. 2003 August; 62(2-3):99-109. Epub 2003 May 13, incorporated by reference in its entirety herein), the type of change (D) (e.g., deletion, promoter swap (“proswp”), start codon swap (“scswp”), replacement (“gene repl”))(most are promoter swaps), the shell number (E), and the shell subclass (F) (e.g., on-pathway, transport, other, TCA, transcription, PTS). Shells 3 and 4 are generally off the biosynthetic pathway. Shell subclass “other” generally corresponds to an unexpected, off-pathway result that may be of interest for further exploration because there is no known biological relationship between the change and the product of interest. Other shell subclasses (some of which are recited in the table of FIGS. 12A-L) are explained below:

on-pathway: on the biosynthetic pathway to the product

transport: ion channels, transporters, and other proteins responsible for transport of molecules in and out of the cell

transcription: transcription factors and other transcriptional regulators

TCA: tricarboxylic acid cycle, also known as the citric acid cycle

PTS: phosphotransferase system, responsible for importing sugars into bacteria

For a particular change (A), the table shows the change in productivity (G) in units of grams/liter/hour and the change in yield (H), the percentage weight ratio in units of grams glucose/grams of product of interest×100.

The promoter (I) identifies the promoter that replaces the native promoter of the gene affected by the change (A). The identifier in the table of the replacement promoter (I) references the gene from which the replacement promoter was derived. If “native” is indicated, then no replacement was made.

The protein names (J) identify the protein made by the gene that was modified (e.g., an enzyme that was increased by a promoter change). Note that the protein made is generally not the product of interest, but rather a protein made by the organism that is affected by the change.

Column K lists the “GO Terms” associated with the genes that were affected by the changes. As discussed elsewhere herein, the GO Terms associated with Shells 3 and 4 are of particular interest for further exploration as high priority targets for potential modification.

A list of the Shell 4 GO Terms from the table of FIGS. 12A-L follows:

de novo CTP biosynthetic process,

3-isopropylmalate dehydratase activity,

4 iron,

4 sulfur cluster binding,

ATP binding,

DNA binding,

DNA topoisomerase activity,

DNA topoisomerase type I activity,

DNA topological change,

DNA-templated,

L-aspartate:2-oxoglutarate aminotransferase activity,

L-phenylalanine:2-oxoglutarate aminotransferase activity,

NADH dehydrogenase activity,

UMP kinase activity,

acetolactate synthase activity,

adenylate cyclase activity,

alcohol dehydrogenase (NAD) activity,

amino acid binding,

aromatic compound biosynthetic process,

biosynthetic process,

branched-chain amino acid biosynthetic process,

cAMP biosynthetic process,

catalytic activity,

cellular amino acid biosynthetic process,

cellular component organization or biogenesis,

cellular macromolecule biosynthetic process,

cellular nitrogen compound biosynthetic process,

cellular process,

chromosome organization,

codon specific,

cyclic nucleotide biosynthetic process,

heterocycle biosynthetic process,

intracellular signal transduction,

ion transport,

iron-sulfur cluster binding,

isomerase activity,

kinase activity,

leucine biosynthetic process,

lyase activity,

metabolic process,

metal ion binding,

nucleotide binding,

nucleotide phosphorylation,

organic acid biosynthetic process,

oxidation-reduction process,

oxidoreductase activity,

phosphorus-oxygen lyase activity,

phosphorylation,

potassium ion transport,

proteolysis,

purine-containing compound metabolic process,

pyridoxal phosphate binding,

pyrimidine nucleotide biosynthetic process,

pyrimidine-containing compound metabolic process,

regulation of cellular biosynthetic process,

regulation of transcription,

sequence-specific DNA binding,

serine-type endopeptidase activity,

signal transducer activity,

signal transduction,

small molecule metabolic process,

transaminase activity,

transcription,

transcription factor activity,

transferase activity,

translation,

translation release factor activity,

translational termination,

transport,

uridylate kinase activity,

DNA metabolic process,

biosynthetic process,

cellular amino acid metabolic process,

metabolic process,

nucleobase-containing compound metabolic process,

translation,

transport.

FIG. 10 illustrates a cloud computing environment according to embodiments of the present disclosure. In embodiments of the disclosure, the prioritization engine software 1010 may be implemented in a cloud computing system 1002, to enable multiple users to prioritize gene modifications according to embodiments of the present disclosure. Client computers 1006, such as those illustrated in FIG. 7, access the system via a network 1008, such as the Internet. The system may employ one or more computing systems using one or more processors, of the type illustrated in FIG. 7. The cloud computing system itself includes a network interface 1012 to interface the software 1010 to the client computers 10010 via the network 1008. The network interface 1012 may include an application programming interface (API) to enable client applications at the client computers 1006 to access the system software 1010. In particular, through the API, client computers 1006 may access the prioritization engine.

A software as a service (SaaS) software module 1014 offers the system software 1010 as a service to the client computers 1006. A cloud management module 10110 manages access to the system 1010 by the client computers 1006. The cloud management module 1016 may enable a cloud architecture that employs multitenant applications, virtualization or other architectures known in the art to serve multiple users.

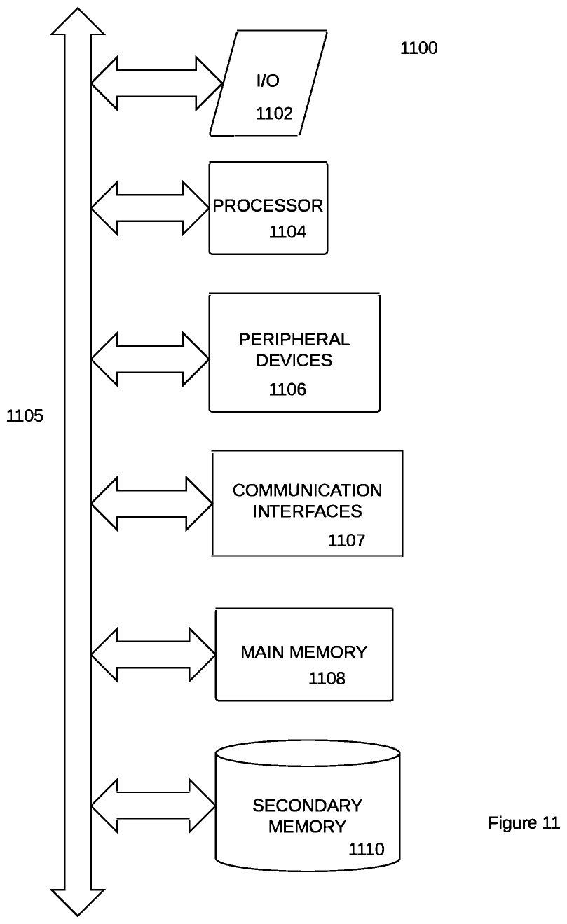

FIG. 11 illustrates an example of a computer system 1100 that may be used to execute program code stored in a non-transitory computer readable medium (e.g., memory) in accordance with embodiments of the disclosure. The computer system includes an input/output subsystem 1102, which may be used to interface with human users and/or other computer systems depending upon the application. The I/O subsystem 1102 may include, e.g., a keyboard, mouse, graphical user interface, touchscreen, or other interfaces for input, and, e.g., an LED or other flat screen display, or other interfaces for output, including application program interfaces (APIs). Other elements of embodiments of the disclosure, such as the prioritization engine may be implemented with a computer system like that of computer system 1100.

Program code may be stored in non-transitory media such as persistent storage in secondary memory 1110 or main memory 1108 or both. Main memory 1108 may include volatile memory such as random access memory (RAM) or non-volatile memory such as read only memory (ROM), as well as different levels of cache memory for faster access to instructions and data. Secondary memory may include persistent storage such as solid state drives, hard disk drives or optical disks. One or more processors 1104 reads program code from one or more non-transitory media and executes the code to enable the computer system to accomplish the methods performed by the embodiments herein. Those skilled in the art will understand that the processor(s) may ingest source code, and interpret or compile the source code into machine code that is understandable at the hardware gate level of the processor(s) 1104. The processor(s) 1104 may include graphics processing units (GPUs) for handling computationally intensive tasks.

The processor(s) 1104 may communicate with external networks via one or more communications interfaces 1107, such as a network interface card, WiFi transceiver, etc. A bus 1105 communicatively couples the I/O subsystem 1102, the processor(s) 1104, peripheral devices 1106, communications interfaces 1107, memory 1108, and persistent storage 1110. Embodiments of the disclosure are not limited to this representative architecture. Alternative embodiments may employ different arrangements and types of components, e.g., separate buses for input-output components and memory subsystems.

Those skilled in the art will understand that some or all of the elements of embodiments of the disclosure, and their accompanying operations, may be implemented wholly or partially by one or more computer systems including one or more processors and one or more memory systems like those of computer system 1100. In particular, the elements of the prioritization engine and any other automated systems or devices described herein may be computer-implemented. Some elements and functionality may be implemented locally and others may be implemented in a distributed fashion over a network through different servers, e.g., in client-server fashion, for example. In particular, server-side operations may be made available to multiple clients in a software as a service (SaaS) fashion, as shown in FIG. 10.

Those skilled in the art will recognize that, in some embodiments, some of the operations described herein may be performed by human implementation, or through a combination of automated and manual means. When an operation is not fully automated, appropriate components of the prioritization engine may, for example, receive the results of human performance of the operations rather than generate results through its own operational capabilities.

### INCORPORATION BY REFERENCE

All references, articles, publications, patents, patent publications, and patent applications cited herein are incorporated by reference in their entireties for all purposes. In particular, this application incorporates by reference U.S. provisional application No. 62/264,232, filed on Dec. 7, 2015, U.S. nonprovisional application Ser. No. 15/140,296, filed on Apr. 27, 2016, and U.S. provisional application No. 62/368,786, filed on Jul. 29, 2016, each of which is hereby incorporated by reference in their entirety.

However, mention of any reference, article, publication, patent, patent publication, and patent application cited herein is not, and should not be taken as an acknowledgment or any form of suggestion that they constitute valid prior art or form part of the common general knowledge in any country in the world, or that they are disclose essential matter.

### Embodiments

1. A computer-implemented method for determining modifications to apply to genes within at least one microbial strain to improve phenotypic performance, the method comprising:
   accessing first phenotypic performance data based at least in part upon first gene modifications made to a first set of genes in at least one microbial strain;
   predicting, using a computing device, second phenotypic performance of second gene modifications, based at least in part upon the first phenotypic performance data and at least one modification feature that is common to the first gene modifications and the second gene modifications; and
   prioritizing, using a computing device, the second gene modifications to be applied to a second set of genes based at least in part upon the second phenotypic performance,
   wherein, based at least in part upon the prioritizing, at least a subset of the second gene modifications may be applied to genes within at least one microbial strain.
2. The method of embodiment 1, wherein the at least one modification feature includes ontological class.
3. The method of any one of embodiments 1 or 2, wherein the at least one modification feature includes gene modification type.
4. The method of embodiment 3, wherein the modification type includes a promoter swap.
5. The method of embodiment 3 or 4, wherein the modification type includes promoter strength of promoter swaps.
6. The method of any one of embodiments 1-5, wherein the predicting more heavily weights medium-strength promoters than strong or weak promoters.
7. The method of any one of embodiments 1-5, wherein the predicting weights weak promoters less heavily than strong and medium-strength promoters.
8. The method of any one of embodiments 3-5, wherein the modification type is a SNP swap.
9. The method of any one of embodiments 1-8, wherein the at least one modification feature includes modifications of one or more types to at least two genes in the at least one strain.
10. The method of any one of embodiments 1-9, wherein the predicting more heavily weights the modifications of one or more types that yield positive epistatic effects.
11. The method of any one of embodiments 1-10, wherein the second set of genes includes no genes within the first set of genes.
12. The method of any one of embodiments 1-11, wherein genes within a subset of genes within the second set of genes are each a member of multiple classes, and predicting second phenotypic performance comprises predicting a composite second phenotypic performance based upon a combination of predicted phenotypic performance for each of the classes to which each gene belongs.
13. The method of any one of embodiments 1-12, wherein genes within the second set of genes share membership in at least one common class, and predicting comprises assigning the same second phenotypic performance to all genes within a common class if the common class is the only class to which such genes belong.
14. The method of any one of embodiments 1-13, wherein genes within the second set of genes are each a member of only a single class.
15. The method of any one of embodiments 1-14, wherein at least one modification feature includes first ontological classes from a first classification system and second ontological classes from a second classification system.
16. The method of any one of embodiments 1-15, wherein the at least one modification feature includes a characteristic of a product synthesized by at least one microbial strain.
17. The method of any one of embodiments 1-16, wherein predicting second phenotypic performance employs genes from the first set of genes as a training set in a machine learning predictive model.
18. The method of any one of embodiments 1-17, wherein
   predicting second phenotypic performance comprises predicting per-class enrichment probabilities for the second gene modifications based at least in part upon the first phenotypic performance data; and
   prioritizing the second gene modifications is based at least in part upon a ranking of the predicted per-class enrichment probabilities.
19. The method of any one of embodiments 1-18, further comprising:
   obtaining updated first phenotypic performance data based at least in part upon application of one or more gene modifications of the second gene modifications to genes within the second set of genes; and
   predicting updated second phenotypic performance of a subset of the second gene modifications, based at least in part upon the updated first phenotypic performance data; and
   prioritizing the subset of the second gene modifications to be applied to a subset of the second set of genes based at least in part upon the updated second phenotypic performance.
20. The method of any one of embodiments 1-19, comprising iteratively updating prioritization of subsets of modifications of the second gene modifications to be applied to subsets of genes within the second set of genes based upon phenotypic performance data obtained from iterative application of one or more gene modifications of the second gene modifications to genes within the second set of genes.
21. The method of any one of embodiments 1-20, wherein the at least one modification feature includes different levels of abstraction within a gene ontology classification.
22. The method of any one of embodiments 1-21, wherein the at least one modification feature includes classification based upon metabolic network.
23. The method of any one of embodiments 1-22, wherein the at least one modification feature relates to at least one microbial strain characteristic.
24. The method of any one of embodiments 1-23, wherein the second set of genes resides within at least one microbial strain different from the at least one microbial strain in which the first set of genes resides.
25. The method of any one of embodiments 1-24, wherein the first phenotypic performance data relates to at least one characteristic of a first product produced by the at least one microbial strain in which the first set of genes reside, and the second phenotypic performance relates to at least one characteristic of a second product that is different from the first product.
26. The method of embodiment 25, wherein the second product is produced by at least one microbial strain different from the at least one microbial strain in which the first set of genes resides.
27. A microbial strain comprising one or more second gene modifications prioritized according to any one of embodiments 1-26.
28. A microbial strain comprising a first gene modification applied to a gene in the first set of genes of any one of embodiments 1-27.
29. The microbial strain of any one of embodiments 1-28, further comprising a second gene modification that is prioritized above a threshold prioritization and applied to at least one gene in the second set of genes.
30. The microbial strain of embodiment 29 wherein the applied gene modification is prioritized higher in response to the prioritization being based on the predicted updated second phenotypic performance than in response to being based on the predicted second phenotypic performance.
31. The method of any one of embodiments 1-30, wherein the at least one modification feature represents at least one of the following ontological classes:
   de novo CTP biosynthetic process,
   3-isopropylmalate dehydratase activity,
   4 iron,
   4 sulfur cluster binding,
   ATP binding,
   DNA binding,
   DNA topoisomerase activity,
   DNA topoisomerase type I activity,
   DNA topological change,
   DNA-templated,
   L-aspartate:2-oxoglutarate aminotransferase activity,
   L-phenylalanine:2-oxoglutarate aminotransferase activity,
   NADH dehydrogenase activity,
   UMP kinase activity,
   acetolactate synthase activity,
   adenylate cyclase activity,
   alcohol dehydrogenase (NAD) activity,
   amino acid binding,
   aromatic compound biosynthetic process,
   biosynthetic process,
   branched-chain amino acid biosynthetic process,
   cAMP biosynthetic process,
   catalytic activity,
   cellular amino acid biosynthetic process,
   cellular component organization or biogenesis,
   cellular macromolecule biosynthetic process,
   cellular nitrogen compound biosynthetic process,
   cellular process,
   chromosome organization,
   codon specific,
   cyclic nucleotide biosynthetic process,
   heterocycle biosynthetic process,
   intracellular signal transduction,
   ion transport,
   iron-sulfur cluster binding,
   isomerase activity,
   kinase activity,
   leucine biosynthetic process,
   lyase activity,
   metabolic process,
   metal ion binding,
   nucleotide binding,
   nucleotide phosphorylation,
   organic acid biosynthetic process,
   oxidation-reduction process,
   oxidoreductase activity,
   phosphorus-oxygen lyase activity,
   phosphorylation,
   potassium ion transport,
   proteolysis,
   purine-containing compound metabolic process,
   pyridoxal phosphate binding,
   pyrimidine nucleotide biosynthetic process,
   pyrimidine-containing compound metabolic process,
   regulation of cellular biosynthetic process,
   regulation of transcription,
   sequence-specific DNA binding,
   serine-type endopeptidase activity,
   signal transducer activity,
   signal transduction,
   small molecule metabolic process,
   transaminase activity,
   transcription,
   transcription factor activity,
   transferase activity,
   translation,
   translation release factor activity,
   translational termination,
   transport,
   uridylate kinase activity,
   DNA metabolic process,
   biosynthetic process,
   cellular amino acid metabolic process,
   metabolic process,
   nucleobase-containing compound metabolic process,
   translation, or
   transport.

1. A computer-implemented method for determining modifications to apply to genes within at least one microbial strain to improve phenotypic performance, the method comprising:
accessing first phenotypic performance data based at least in part upon first gene modifications made to a first set of genes in at least one microbial strain;
predicting, using a computing device, second phenotypic performance of second gene modifications, based at least in part upon the first phenotypic performance data and at least one modification feature that is common to the first gene modifications and the second gene modifications; and
prioritizing, using a computing device, the second gene modifications to be applied to a second set of genes based at least in part upon the second phenotypic performance,
wherein, based at least in part upon the prioritizing, at least a subset of the second gene modifications may be applied to genes within at least one microbial strain.

2. The method of claim 1, wherein the at least one modification feature includes ontological class.

3. The method of claim 1, wherein the at least one modification feature includes gene modification type.

4. The method of claim 1, wherein, based at least in part upon the prioritizing, at least a subset of the second gene modifications is applied to genes within at least one microbial strain.

5. (canceled)

6. The method of claim 3, wherein the gene modification type includes a promoter swap, and the predicting more heavily weights medium-strength promoters than strong or weak promoters.

7. (canceled)

8. (canceled)

9. The method of claim 1, wherein the at least one modification feature includes modifications of one or more types to at least two genes in the at least one strain.

10. The method of claim 9, wherein the predicting more heavily weights the modifications of one or more types that yield positive epistatic effects.

11. The method claim 1, wherein the second set of genes includes no genes within the first set of genes.

12. (canceled)

13. (canceled)

14. (canceled)

15. (canceled)

16. The method of claim 1, wherein the at least one modification feature includes a characteristic of a product synthesized by at least one microbial strain.

17. The method of claim 1, wherein predicting second phenotypic performance employs genes from the first set of genes in a training set in a machine learning predictive model.

18. The method of claim 1, wherein
predicting second phenotypic performance comprises predicting per-class enrichment probabilities for the second gene modifications based at least in part upon the first phenotypic performance data; and
prioritizing the second gene modifications is based at least in part upon a ranking of the predicted per-class enrichment probabilities.

19. The method of claim 1, further comprising:
obtaining updated first phenotypic performance data based at least in part upon application of one or more gene modifications of the second gene modifications to genes within the second set of genes; and
predicting updated second phenotypic performance of a subset of the second gene modifications, based at least in part upon the updated first phenotypic performance data; and
prioritizing the subset of the second gene modifications to be applied to a subset of the second set of genes based at least in part upon the updated second phenotypic performance.

20. (canceled)

21. (canceled)

22. The method of claim 1, wherein the at least one modification feature includes classification based upon metabolic network.

23. (canceled)

24. The method of claim 1, wherein the second set of genes resides within at least one microbial strain different from the at least one microbial strain in which the first set of genes resides.

25. (canceled)

26. (canceled)

27. A microbial strain comprising one or more second gene modifications prioritized by the method of claim 1.

28. (canceled)

29. (canceled)

30. (canceled)

31. (canceled)

32. A system for determining modifications to apply to genes within at least one microbial strain to improve phenotypic performance, the system comprising:
one or more memories storing program code; and
one or more processors, operatively coupled to the one or more memories, for executing the program code to cause performance of:
accessing first phenotypic performance data based at least in part upon first gene modifications made to a first set of genes in at least one microbial strain;
predicting, using a computing device, second phenotypic performance of second gene modifications, based at least in part upon the first phenotypic performance data and at least one modification feature that is common to the first gene modifications and the second gene modifications; and
prioritizing, using a computing device, the second gene modifications to be applied to a second set of genes based at least in part upon the second phenotypic performance,
wherein, based at least in part upon the prioritizing, at least a subset of the second gene modifications may be applied to genes within at least one microbial strain.

33. The system of claim 32, wherein, based at least in part upon the prioritizing, at least a subset of the second gene modifications is applied to genes within at least one microbial strain.

34. The system of claim 32, wherein the one or more memories further store program code, the execution of which causes performance of:
obtaining updated first phenotypic performance data based at least in part upon application of one or more gene modifications of the second gene modifications to genes within the second set of genes; and
predicting updated second phenotypic performance of a subset of the second gene modifications, based at least in part upon the updated first phenotypic performance data; and
prioritizing the subset of the second gene modifications to be applied to a subset of the second set of genes based at least in part upon the updated second phenotypic performance.

35. One or more non-transitory computer-readable media storing program code for determining modifications to apply to genes within at least one microbial strain to improve phenotypic performance, wherein the program code, when executed by one or more processors, causes performance of:
accessing first phenotypic performance data based at least in part upon first gene modifications made to a first set of genes in at least one microbial strain;
predicting, using a computing device, second phenotypic performance of second gene modifications, based at least in part upon the first phenotypic performance data and at least one modification feature that is common to the first gene modifications and the second gene modifications; and
prioritizing, using a computing device, the second gene modifications to be applied to a second set of genes based at least in part upon the second phenotypic performance,
wherein, based at least in part upon the prioritizing, at least a subset of the second gene modifications may be applied to genes within at least one microbial strain.

36. The one or more non-transitory computer-readable media of claim 35, wherein, based at least in part upon the prioritizing, at least a subset of the second gene modifications is applied to genes within at least one microbial strain.

37. The one or more non-transitory computer-readable media of claim 35 further storing program code, which, when executed, causes performance of:
obtaining updated first phenotypic performance data based at least in part upon application of one or more gene modifications of the second gene modifications to genes within the second set of genes; and
predicting updated second phenotypic performance of a subset of the second gene modifications, based at least in part upon the updated first phenotypic performance data; and
prioritizing the subset of the second gene modifications to be applied to a subset of the second set of genes based at least in part upon the updated second phenotypic performance.

38. A computer-implemented method for prioritizing genetic modifications applied to genes within at least one microbial strain, the method comprising:
accessing a prioritization of candidate gene modifications, wherein
the prioritization is based at least in part upon predicted phenotypic performance of the candidate gene modifications,
the predicted phenotypic performance is based at least in part upon observed phenotypic performance of first gene modifications within at least one first microbial strain, and
a subset of the candidate gene modifications is applied to genes within at least one second microbial strain.

39. The method of claim 38, wherein the first gene modifications relate to a first set of genes, the candidate gene modifications relate to a second set of genes, and the predicted phenotypic performance is also based at least in part upon at least one modification feature that is common to the first gene modifications and the second gene modifications.

40. A system for prioritizing genetic modifications applied to genes within at least one microbial strain, the system comprising:
one or more memories storing program code; and
one or more processors, operatively coupled to the one or more memories, for executing the program code to cause performance of:
accessing a prioritization of candidate gene modifications, wherein
the prioritization is based at least in part upon predicted phenotypic performance of the candidate gene modifications,
the predicted phenotypic performance is based at least in part upon observed phenotypic performance of first gene modifications within at least one first microbial strain, and
a subset of the candidate gene modifications is applied to genes within at least one second microbial strain.

41. The system of claim 40, wherein the first gene modifications relate to a first set of genes, the candidate gene modifications relate to a second set of genes, and the predicted phenotypic performance is also based at least in part upon at least one modification feature that is common to the first gene modifications and the second gene modifications.

42. One or more non-transitory computer-readable media storing program code for prioritizing genetic modifications applied to genes within at least one microbial strain, wherein the program code, when executed by one or more processors, causes performance of:
accessing a prioritization of candidate gene modifications, wherein
the prioritization is based at least in part upon predicted phenotypic performance of the candidate gene modifications,
the predicted phenotypic performance is based at least in part upon observed phenotypic performance of first gene modifications within at least one first microbial strain, and
wherein a subset of the candidate gene modifications is applied to genes within at least one second microbial strain.

43. The one or more non-transitory computer-readable media of claim 42, wherein the first gene modifications relate to a first set of genes, the candidate gene modifications relate to a second set of genes, and the predicted phenotypic performance is also based at least in part upon at least one modification feature that is common to the first gene modifications and the second gene modifications.
